(19) United States
(12) Patent Application Publication
(10) Pub. No.: US 2011/0151457 A1
(43) Pub. Date: Jun. 23, 2011

(54) HYPERTHEROMOSTABLE ENDONUCLEASE IV SUBSTRATE PROBE

(75) Inventors: Yevgeniy Belousov, (US); Eugeny Lukhtanov, (US)

(73) Assignee: Elitech Holding B.V.

(21) Appl. No.: 12/970,344

(22) Filed: Dec. 16, 2010

Related U.S. Application Data
(60) Provisional application No. 61/289,152, filed on Dec. 22, 2009.

(51) Int. Cl. C12Q 1/68; C07H 21/04

(52) U.S. Cl. 435/6.11; 536/24.3

(57) ABSTRACT

The present invention relates to a hyperthermostable endonuclease IV substrate probe to be used in nucleic acid assay methods which can be carried out using hyperthermostable enzymes, including detection of target nucleic acids, and detection of nucleic acid polymorphism.

This application claims priority to U.S. Provisional Patent Application Ser. No. 61/289,152, entitled “Hyperthermostable Endonuclease IV Substrate Probe,” filed on Dec. 22, 2009, the entire content of which is hereby incorporated by reference.

### FIELD OF THE INVENTION

The present invention relates to cleavable probes for use in nucleic acid assays, more specifically to a hyperthermostable endonuclease IV substrate probe capable of being cleaved by a hyperthermostable endonuclease IV.

### BACKGROUND

Polynucleotide identification assays that are based on the selective cleavage of a probe hybridized to a target nucleic acid have been disclosed. U.S. Pat. Nos. 5,656,430; 5,763,178; and 6,340,566 disclose methods for detecting point mutations by using an endonuclease to cleave the nucleic acid backbone in the middle of the oligonucleotide at the point of mutation. In methods that identify a mismatch by enzymatic cleavage of a nucleic acid backbone, the presence, rather than the absence, of a mismatch stimulates the cleavage of the probe phosphodiester backbone.

In some cases, polynucleotide identification assays rely on the creation of an artificial apurinic/apyrimidinic (AP), or abasic, site, and the subsequent cleavage by an enzyme which specifically recognizes AP sites. AP sites arise spontaneously in DNA, and are cytotoxic and mutagenic and need to be repaired quickly in order to maintain the functional and genetic integrity of the genome.

AP sites in double-stranded DNA are recognized by a class of enzymes termed Class II AP endonucleases that cleave the phosphodiester backbone on the 5′ side of the AP site via a hydrolytic mechanism, thereby providing a free 3′-OH group that serves as a substrate for DNA polymerases to initiate Base Excision Repair (BER). The endonuclease IV from Escherichia coli (E. coli) is one example of a Class II AP endonuclease (see Weiss, B., 1998).

U.S. Pat. No. 5,955,268 discloses the cleavage of an immobilized-abasic-site containing probe which is cleaved when hybridized to its complementary target.

U.S. Pat. Nos. 5,516,663 and 5,792,607 disclose using endonuclease IV isolated from E. coli to remove an abasic site incorporated as a blocking agent on the 3′ end of an oligonucleotide to improve specificity and sensitivity of the ligase chain reaction (LCR) or polymerase chain reaction (PCR) amplification.

As described above, many polynucleotide identification assays rely on endonuclease IV isolated from E. coli, which does not have thermostable properties. Therefore, the isolated E. coli endonuclease VI is not stable in DNA amplification reactions.

The need for thermostable or hyperthermostable enzymes is frequently an obstacle in various laboratory reactions including amplification reactions. One means of obtaining thermostable or hyperthermostable enzymes is by isolating the required enzyme from a thermophile or hyperthermophile, respectively, which grows optimally at higher than ambient temperatures.

International Patent Publication WO 93/20191 discloses a recombinant class II apurinic endonuclease having substantially no exonuclease activity and which retains activity when subjected to elevated temperatures for the time necessary to effect denaturation of double stranded nucleic acids. Moderately stable endonuclease IV enzymes have been reported isolated from Thermothrix thiopara (Kaboev, O. K., 1985), E. coli and Thermus sp. strain X-1 cells (Warner, H. R., 1983)) and Bacillus stearothermphilus (Bibor, V, and Verly, W. G., 1978). Endonuclease IV from Thermotoga maritima denatures only at temperatures approaching 90° C. (Haas, B. J., 1999).

U.S. Pat. No. 7,252,940 discloses a method of detecting a target nucleic acid using an AP probe labeled at the 5′-end with a functional tail, which tail is cleaved on hybridization of the probe to its complementary target by an AP endonuclease isolated from Escherichia coli, is hereby incorporated by reference. The cleaved tail R is detected during or after the cleavage reaction is completed. In a preferred embodiment, the AP endonuclease cleavage is facilitated by the inclusion of an enhancer.

Hyperthermophiles grow optimally at temperatures between 80° C. and 110° C. in contrast to thermophiles which grow optimally between 60° C. and 80° C. (Vielle and Zeikus, 2001, hereby incorporated by reference). Hyperthermophiles are listed in Table 1 with their optimum growth temperature. Due to their stability at increased temperatures compared with E. Coli, enzymes isolated from hyperthermophiles can be used in assays requiring a variety of temperatures, without becoming denatured and losing their activity.

TABLE 1
Hyperthermophiles and Their Optimum Growth Temperature

| | Optimum Growth |
|---|---|
| Organisms | Temperature ° C. |
| Aquifex pyrophilus | 85 |
| Thermocrinus rubber | 80 |
| Thermotoga maritime | 80 |
| Thermotago strain FjSS3-B1 | 80-85 |
| Sulfolobus shibatae | 81 |
| S. solfataricu | 87 |
| Stygiolabus azoricus | 80 |
| Acidianus infernus | 90 |
| A. ambivalens | 80 |
| Thermoproteus tenax | 88 |
| T. neurtophilus | 85 |
| T. uzoniensis | 90 |
| Pyrobaculum islandicum | 100 |
| P. organotrophum | 102 |
| P. aerophilum | 100 |
| Thermofilum pendens | 85-90 |
| Desulfurococcus mobilis | 85 |
| D. amylolyticus | 90-92 |
| Staphylothermus marinus | 92 |
| Thermosphaera aggregans | 85 |
| Pyrodictium occultum | 105 |
| P. abyssi | 97 |
| P. prockii | 105 |
| Hyperthermus butylicus | 95-106 |
| Thermodiscus maritimus | 85 |
| Pyrolobus fumarii | 106 |
| Aeropyrum pernix | 90-95 |
| Caldococcus litoralis | 88 |
| Palaeococcus ferrophilus | 83 |
| Thermococcus aggregans | 88 |
| T. barophilus | 85 |
| T. guaymasensis | 88 |
| T. celler | 88 |
| T. acidaminovorans | 85 |
| T. chitonophagus | 85 |
| T. barossii | 82.5 |
| T. litoralis | 85 |
| T. profundus | 80 |
| T. hydrothermalis | 85 |
| Pyrococcus furiosus | 100 |
| P. woesei | 100-103 |
| P. abyssi | 96 |
| P. horikoshii | 98 |
| Archaeoglobus fulgidus | 83 |
| A. profundus | 82 |
| Methanococcus jannaschii | 85 |
| M. valcanius | 80 |
| M. vervens | 85 |
| M. igneus | 88 |
| M. infemus | 85 |
| Methanothermus fervidus | 83 |
| M. sociabilis | 88 |
| Methanopyrus kandleri | 98 |

Hyperthermostable endonuclease IV enzyme has been isolated from Thermotoga maritima (Haas, B. J., 1999), and Pyrobaculum aerophilum (Sartori & Jiricny, 2003). A versatile endonuclease IV from Thermus thermophilus has uracil-excising and 3′-5′ exonuclease activity (Back et al., 2006; International Patent Publication No. WO 93/20191).

It was reported in 2001 that more than 100 genes for hyperthermopholic enzymes have been cloned and expressed in mesophiles. In addition it was observed that less than 10% of all hyperthermopholic enzymes expressed in E. coli, have stability, catalytic or structural properties different from the enzymes purified from the native organism (Vielle & Zeikus, 2001).

Protein thermostability engineering has shown that protein stability can be enhanced without deleterious effect on activity and that actual stability and activity can be increased simultaneously (Giver et al., 1998; Van den Berg et al., 1998). Direct evolution is an established method of designing enzymes with increased stability (Veile and Zeikus, 1999). A number of computer algorithms based on physical and chemical principles are used to predict protein rigidity and stability to design and developed stabilizing mutations (Veile and Zeikus, 1998).

Some thermophilic enzymes have been described that contain metal atoms that are not present in their mesophile homologs and that some studies observations suggest major stabilizing forces associated with metal ions in the holoenzyme. Metal ions known to play a role in the stabilization of thermophilic proteins include Mg²⁺, Co²⁺, Mn⁺, Ca²⁺, and Zn²⁺.

What is needed in the art is a hyperthermostable endonuclease substrate probe capable of being used in polynucleotide identification assays. A hyperthermostable endonuclease probe, together with a hyperthermostable endonuclease, could be used in combination with amplification or other reactions requiring high temperatures. Such amplification reactions could then be carried out homogenously, without requiring additional endonuclease following a heating step.

### SUMMARY

The present invention relates to a hyperthermostable endonuclease IV substrate probe to be used in a nucleic acid assay. In an embodiment of the invention, the hyperthermostable endonuclease IV substrate probe may comprise a nucleic acid probe comprised of an oligonucleotide sequence attached at a 3′ end via a phosphodiester bond of a phosphate group, to a functional, chemical tail comprising a hyperthermostable endonuclease IV cleavage site.

In one embodiment, the nucleic acid probe is comprised of an olignucleotide sequence NA attached at a 3′ end via a phosphodiester bond of a phosphate group, to a functional, chemical tail R comprising a hyperthermostable endonuclease VI cleavage site.

In another embodiment, the nucleic acid probe is comprised of an oligonucleotide sequence NA attached at a 3′ end via a phosphodiester bond of a phosphate group, to a functional, chemical tail through a linker L that allows specific cleavage by a hyperthermostable endonuclease VI.

In another embodiment, the nucleic acid probe is comprised of an oligonucleotide sequence NA attached at a 3′ end via a phosphodiester bond of a phosphate group, to a functional, chemical tail R, which comprises LR′, wherein L is a linker, and R′ is a functional, chemical tail. The functional, chemical tail R can be a reporter moiety or a quencher moiety, or can be an L-linked-reporter or a L-linked-quencher moiety, wherein L is a linker.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a diagram of a endonuclease IV cleavable probe in an embodiment of the present invention;

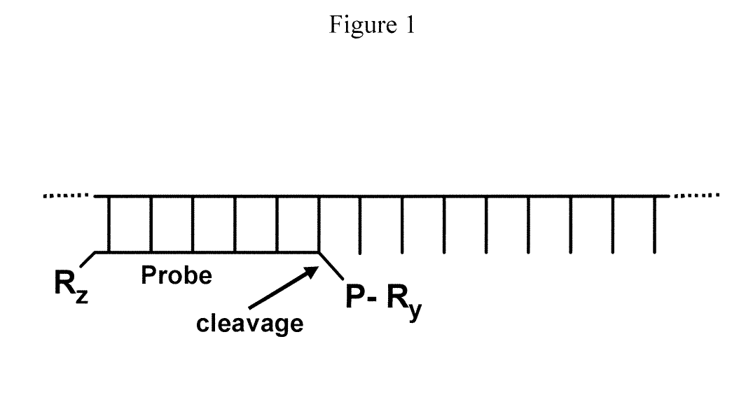

FIG. 2 shows a closed tube PCR amplification followed by post PCR Endonuclease IV detection of 1 ng M. tuberculosis with the probe containing linker 6 (see Table 2), in an embodiment of the present invention;

FIG. 3 shows an example of inhibition of the Tth Endonuclease IV cleavage by the enhancer, in an embodiment of the present invention;

FIG. 4 shows an example of detection of the G and A alleles in closed-tube format in PCR synthetic templates, in an embodiment of the present invention. a) shows the detection of the wild type allele “A” in the FAM-channel with wild-type specific probe; and b) shows the detection of the mutant allele “A” in the YY-channel with mutant specific probe;

FIG. 5 shows an example of scatter plot analysis of a SNP with the probes specific for wild type and mutant alleles, in an embodiment of the present invention;

FIG. 6 shows an example of FAM-solid support 15 and phosphoramidites 16 to 21 used in the automated synthesis of labeled oligonucleotides, in an embodiment of the present invention;

FIG. 7 shows an example of phosphoramidites 22 to 28 used in the automated synthesis of labeled oligonucleotides, in an embodiment of the present invention; and

FIG. 8 shows a comparison of the change in relative signal fluorescence of match and different mismatches at different positions in a 14-mer probe in an Endo IV assay run at 55° C., in an embodiment of the present invention.

### DETAILED DESCRIPTION OF EMBODIMENTS OF THE INVENTION

The present invention relates to an endonuclease IV substrate probe, and nucleic acid assay methods which can be carried out using hyperthermostable enzymes. In one embodiment, the invention provides a nucleic acid assay using endonuclease IV isolated from a hyperthermophile, for example, Thermus thermophilus.

In one embodiment, the present invention provides an endonuclease IV substrate probe comprising an oligonucleotide sequence NA, attached via a phosphate moiety to a linker L and a functional, chemical tail R. The endonuclease IV substrate probe may be specifically cleaved by endonuclease IV isolated from a hyperthermophile, for example, Thermus thermophilus.

The present invention further encompasses a method for detection of a nucleic acid sequence using a hyperthermostable endonuclease IV substrate probe comprising an oligonucleotide sequence NA, attached via a phosphate moiety to a linker L and a functional, chemical tail R. The endonuclease IV substrate probe may be specifically cleaved by endonuclease IV isolated from a hyperthermophile, for example, Thermus thermophilus. In some embodiments, the method for detection of a nucleic acid sequence may further comprise the use of a metal ion or a detergent in a reaction mixture including the target sequence and the hyperthermostable endonuclease IV substrate probe.

In one embodiment, the endonuclease IV substrate probe of the present invention maybe used in real-time amplification and post-amplification methods without requiring the addition of primers, additional enzymes other than the polymerase, or additional steps. In some embodiments, the real-time amplification and post-amplification methods may further comprise the use of a metal ion or a detergent in a reaction mixture including the target sequence and the hyperthermostable endonuclease IV substrate probe.

### I DEFINITIONS

As used herein, an “endonuclease IV substrate probe” refers to a nucleic acid probe capable of recognizing a target sequence, and comprising a functional, chemical tail which can be cleaved by the endonuclease IV enzyme. In some embodiments, the endonuclease IV enzyme may be derived from a hyperthermophile, for example, Thermus thermophilus. An endonuclease IV substrate probe may comprise an oligonucleotide sequence NA attached at a 3′ end via a phosphodiester bond of a phosphate group P, to a functional, chemical tail R. For example, a nucleic acid probe may comprise an oligonucleotide sequence NA attached at a 3′ end via a phosphodiester bond of a phosphate group, to a functional, chemical tail through a linker L that allows specific cleavage. In another example, a nucleic acid probe may comprise an oligonucleotide sequence NA attached at a 3′ end via a phosphodiester bond of a phosphate group, to a functional, chemical tail R, which comprises LR′, wherein L is a linker, and R′ is a functional, chemical tail. The functional, chemical tail R can be a reporter moiety or a quencher moiety, or can be an L-linked-reporter or a L-linked-quencher moiety, wherein L is a linker.

As used herein, the term “homogeneous amplification” refers to amplification and detection of nucleic acids without the requirement of adding additional reagents or solvents to the reaction mixture. In one example, homogenous amplification can be carried out without opening a reaction vessel, or can be carried out in a sealed reaction vessel, such as a sealed tube.

As used herein, the term “thermostable” refers to an enzyme that retains activity on exposure to temperatures up to about 80° C. For example, a thermostable enzyme would retain activity when exposed to polymerase chain reaction thermocycling conditions involving denaturation steps carried out in the range of 60° C. and 80° C. As used herein, a thermostable enzyme has thermostability in the range of about 60° C. and 80° C.

As used herein, the term “hyperthermostable” refers to an enzyme that retains activity on exposure to temperatures up to about 110° C. For example, a hyperthermostable enzyme would retain activity when exposed to polymerase chain reaction thermocycling conditions involving denaturation steps carried out in the range of 80° C. and 110° C. As used herein, a hyperthermostable enzyme has thermostability in the range of about 80° C. and 110° C.

As used herein, the term “endonuclease IV” refers to an enzyme capable of acting on oxidative damage in DNA. Endonuclease IV may hydrolyse apurinic/apyrimidinc (AP) sites in a nucleic acid strand. Endonuclease may be isolated from E. Coli, or may be isolated from thermostable or hyperthermostable organisms, for example from Thermotoga maritime, Pyrobaculum aerophilum, or Thermus thermophilus, Endonuclease IV may cleave the phosphodiester backbone of a DNA sequence, and may provide a free 3′-OH group that serves as a substrate for DNA polymerases.

### II DESCRIPTION OF THE EMBODIMENTS

### Target Nucleic Acid

Probes comprising a nucleic acid, an endonuclease IV cleavage site and a functional tail are useful for the detection of single-stranded nucleic acids (“ssNA”) and double-stranded nucleic acids (“dsNA”). When used for the detection of double-stranded nucleic acids, unless the population of dsNA contains a sufficient amount of ssNA to be detected using an endonuclease IV cleavage site probe, the dsNA is prepared to provide a sufficient amount of ssNA. Ordinarily, the dsNA is melted or denatured at an elevated temperature prior to their detection. Also, dsNA can be prepared such that a fragment of the target nucleic acids to which the probe is complimentary is single-stranded while the rest of the target is double-stranded. Also, ssNA can be prepared by a preferential amplification of one of the strands of the dsNA. Single-stranded target nucleic acids can be isolated from the double-stranded forms using available molecular biology or physicochemical methods, including strand-specific enzymatic degradation, limited digestion of the double-stranded target followed by heat treatment, or affinity capture through a sequence-incorporated affinity label followed by heat-induced separation from the complementary strand.

Target nucleic acids can be isolated from a variety of natural sources, including blood, homogenized tissue, fixed tissue, tumor biopsies, stool, clinical swabs, food products, hair, plant tissues, microbial culture, public water supply, amniotic fluid, urine, or the like. Techniques useful for the detection of isolated target nucleic acids include, for example, amplification techniques, e.g., polymerase chain reaction (PCR), Mullis, U.S. Pat. No. 4,683,202; ligase-based techniques, e.g., reviewed by Barany, PCR Methods and Applications 1: 5-16 (1991); strand-displacement amplification, Walker et al., U.S. Pat. No. 5,422,252; reverse transcriptase-based techniques, e.g., Davey et al., U.S. Pat. No. 5,409,818; Q.beta. replicase-based techniques, e.g., Chu et al., U.S. Pat. No. 4,957,858; branched DNA techniques, Urdea et al., U.S. Pat. No. 5,124,246; techniques employing RNA-DNA chimeric probes, Duck et al., U.S. Pat. No. 5,011,769; and the like.

Samples containing target nucleic acids can be isolated from natural sources or provided as result of any known method in the art. The target nucleic acid can be cloned, synthetic, or natural. The target nucleic acid can be deoxyribonucleic acid (DNA), including genomic DNA or cDNA, or ribonucleic acid (RNA). Usually a DNA target nucleic acid is preferred. Target nucleic acids can be of diverse origin, including mammalian, bacterial, fungal, viral, or plant origin. The need for extraction, purification, or isolation steps depends on several factors, including the abundance of the target nucleic acids in the sample, the nature of the target nucleic acids, e.g., whether it is RNA or DNA, the presence of extraneous or associated material such as cell walls, histones, or the like, the presence of enzyme inhibitors, and so forth.

Guidance for selecting an appropriate protocol for particular applications for extraction, purification and/or isolation of target nucleic acids can be found in, for example, Chen and Janes, Editors, PCR Cloning Protocols (Humana Press, Totowa, N.J., 2002); Sambrook et al., Molecular Cloning, Second Edition (Cold Spring Harbor Laboratory Press, 2001); White, Editor, PCR Cloning Protocols: from molecular cloning to genetic engineering (Humana Press, Totowa, N.J., 1997); Methods in Enzymology, Volumes 6 and 12, parts A and B (Academic Press, New York); McPherson et al., Editors, PCR: A Practical Approach (IRL Press, Oxford, 1991); Herrington et al., Editors, Diagnostic Molecular Pathology: A Practical Approach, Vol. 1 & 2 (IRL Press, Oxford, 1992); Innis, et al., Editors, PCR Protocols (Academic Press, San Diego, 1990); and the like. Typically, preparation protocols involve the application of chaotropic agents, for example, low molecular weight ionic compounds, that favor the solubilization of hydrophobic substances, chelating agents (for instance, EDTA), to disable nucleases, proteases to disable nucleases, detergents, pH buffers, and the like, that serve to isolate and/or protect nucleic acids. Optionally, samples can be treated to reduce the size of the target nucleic acids, such as by sonication, nuclease treatment, or the like. After such initial preparation steps, preferably a sample is treated to denature, i.e. render single-stranded, the target polynucleotide prior to exposing it to the hyperthermostable endonuclease IV substrate probe and hyperthermostable endonuclease IV in accordance with the invention. Preferably, denaturation is achieved by heating the sample at 93° C. to 95° C. for five minutes.

In assays of the present invention, a target nucleic acid is typically included at a concentration of about 2-10 nM, more typically about 4-8 nM, and preferably at a concentration of about 5 nM. However, one of skill in the art will appreciate that the invention is not so limited and other concentrations of target can also be used, whether higher or lower than those indicated above. It is further contemplated that an assay as described herein would be functional with one copy of the target nucleic acid per reaction.

In some embodiments of the present invention, the target nucleic acid includes a diagnostic target, a drug target, a differentiation target subtype, a genetic-based disease marker, a drug activity marker, an oncogene, or any known gene or mutated gene providing information about clinical status. For example, the target nucleic acid may include wildtype or mutated forms of nucleic acids relating to HIV1, HIV2, cancer biomarkers, p450 drug metabolizing enzymes, growth factors, foreign DNA markers, BRCA-1, BRCA-2, abl, abl/bcr, Af4/hrx, akt-2, alk, ALK/NPM, aml1 aml1/mtg8, axl, bcl-2, bcl-3, bcl-6, bcr/abl, c-myc, dbl, dek/can, E2A/pbx1, egfr, enl/hrx, erg/c16, erbB, erbB-2, neu, TSC2,trk Tiam-1 tan-1 tal-1, tal-2, Src, set/can, sis, ski, ros, rhom-1, rhom-2, ret, rel/nrg, rasN, rasK, rash, RAR/PML, raf, PRAD-1, PMS1, PMS2, PML/RAR, pim-1, pbx1/E2A, pax-5, ost, nrg/rel, NPM/ALK, N-myc, neu, erb-2, YH11/CBFB, myb, mtg8/aml1, MSH2, mos, MLM, mll, MLH1, mdm-2, mas, lt-10/C alpha1, lyt-10, lyl-1, L-myc, lmo-1, 2, lck, Lbc, K-sam, KS3, kit, jun, int-2, IL-3, hst, hrx/af4, hrx/enl, hox11, HER2/neu, gsp, gli, gip, fps, fos, fins, ews/fli-1, ets-1, or any combination thereof.

### Hyperthermostable Endonuclease IV Substrate Probe

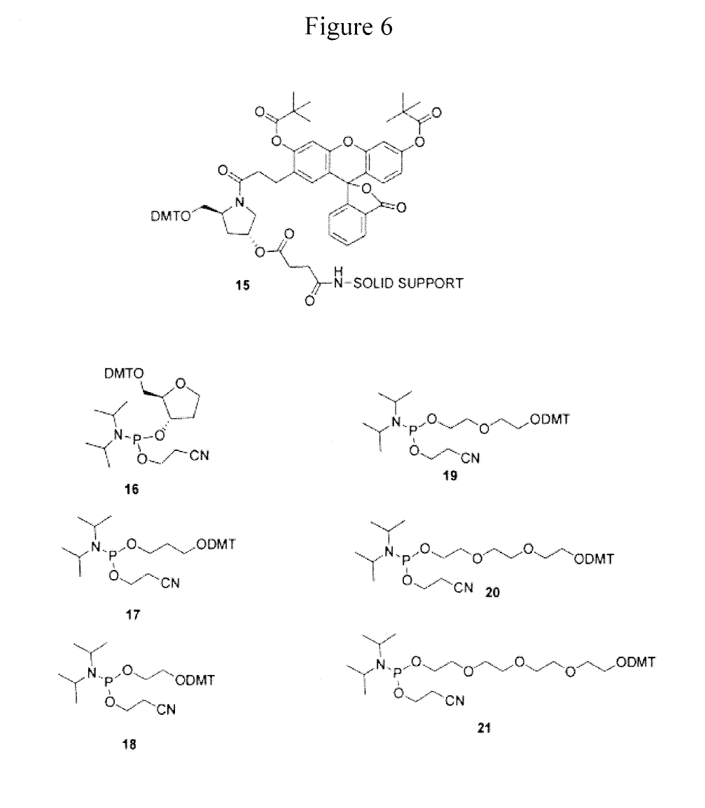

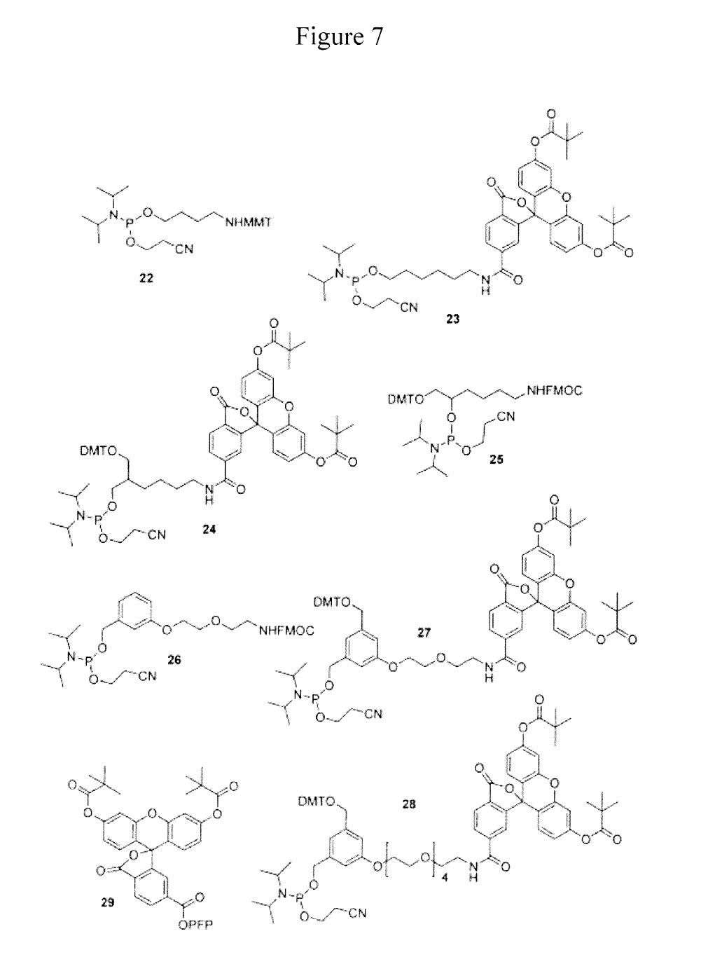

In an embodiment of the invention, a hyperthermostable endonuclease IV substrate probe was synthesized using a commercial oligonucleotide synthesizer using solid support, nucleoside phosphoramidites, phosphoramidite linkers, quencher phosphoramidites and fluorophore phosphoramidites. Hyperthermostable endonuclease IV substrate probes may be synthesized using any method known in the art. In some embodiments the fluorophore was introduced to the hyperthermostable endonuclease IV substrate probe using post-synthesis modification. Examples of linker phosphoramidites used to produce some of the probes disclosed in Table 2 are shown in FIGS. 6 and 7.

In one embodiment, the hyperthermostable endonuclease IV substrate probe comprises a nucleic acid probe comprised of an oligonucleotide sequence attached at a 3′ end via a phosphodiester bond of a phosphate group, to a functional, chemical tail comprising a hyperthermostable endonuclease IV substrate.

In one embodiment, the nucleic acid probe is comprised of an olignucleotide sequence NA attached at a 3′ end via a phosphodiester bond of a phosphate group, to a functional, chemical tail R comprising a hyperthermostable endonuclease IV substrate.

In another embodiment, the nucleic acid probe is comprised of an oligonucleotide sequence NA attached at a 3′ end via a phosphodiester bond of a phosphate group, to a functional, chemical tail through a linker L that allows specific cleavage by a hyperthermostable endonuclease IV.

In another embodiment, the nucleic acid probe comprises an oligonucleotide sequence NA attached at a 3′ end via a phosphodiester bond of a phosphate group, to a functional, chemical tail R, which comprises LR′, wherein L is a linker, and R′ is a functional, chemical tail. The functional, chemical tail R can be a reporter moiety or a quencher moiety, or can be an L-linked-reporter or a L-linked-quencher moiety, wherein L is a linker.

In one embodiment, the fluorophore is attached to the 5′ end of the oligonucleotide NA. In another embodiment, the quencher is attached to an interior base of the oligonucleotide NA.

In yet another embodiment, the hyperthermostable endonuclease IV substrate probe has the general structure:

5′-Quencher-NA-O—P(═O)(O—)-L-Fluorophore

wherein NA is an oligonucleotide sequence as described herein, and L is a linker as described herein.

In yet another embodiment, the hyperthermostable endonuclease IV substrate probe has the general structure:

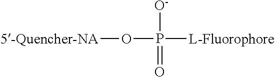

wherein NA is an oligonucleotide sequence as described herein, and L is a linker as described herein.

In an alternative embodiment, the hyperthermostable endonuclease IV substrate probe has the general structure:

5′-Fluorophore-NA-O—P(═O)(O—)-L-Quencher

wherein NA is an oligonucleotide sequence as described herein, and L is a linker as described herein.

Moreover, a fluorophore or quencher as described in any of the above embodiments may be located at the 5′ position of the oligonucleotide sequence NA, or at the 3′ position, or at any position within the oligonucleotide sequence NA.

Oligonucleotide Sequence (“NA”) Component of the Hyperthermostable Endonuclease IV Substrate Probe

The number of nucleotides in the NA component can be 3 to 200, 3 to 100 or 3 to 50 nucleotides in length, depending on the intended use. Usually, the length of the NA is from 5 to 30 nucleotides. More typically, the length of the NA is 6-25, 7-20, or 8-17 nucleic acids. Most often, the NA component is about 6, 7, 8, 9, 10, 11, 12, 13, 14, 15 or 16 nucleic acids in length. Usually, the NA component will have a hybridization melting temperature of about 10° C. to 80° C., more typically of about 20° C. to 70° C., and preferably about 30° C., 40° C., 50° C., 55° C. or 60° C.

The sugar, or glycoside, portion of the NA component of the conjugates can comprise deoxyribose, ribose, 2-fluororibose, and/or 2-O-alkyl or alkenylribose wherein the alkyl group comprises 1 to 6 carbon atoms and the alkenyl group comprises 2 to 6 carbon atoms. In the naturally-occurring nucleotides, modified nucleotides and nucleotide analogues that can comprise an oligonucleotide, the sugar moiety forms a furanose ring, the glycosidic linkage is of the beta configuration, the purine bases are attached to the sugar moiety via the purine 9-position, the pyrimidines via the pyrimidine 1-position and the pyrazolopyrimidines via the pyrazolopyrimidine 1-position (which is equivalent to the purine 9-position). In a preferred embodiment, the sugar moiety is 2-deoxyribose; however, any sugar moiety known to those of skill in the art that is compatible with the ability of the oligonucleotide portion of the compositions of the invention to hybridize to a target sequence can be used.

In one preferred embodiment, the NA is DNA. A hyperthermostable endonuclease IV substrate probe comprising DNA can be used to detect DNA, as well as RNA, targets. In another embodiment, the NA is RNA. A hyperthermostable endonuclease IV substrate probe comprising RNA is generally used for the detection of target DNAs. In another embodiment, a hyperthermostable endonuclease IV substrate probe can contain both DNA and RNA distributed within the probe. In mixed nucleic acid probes, DNA bases preferably are located at 3′-end of the probe while RNA bases are at the 5′-end. It is also preferred when the 3′-terminal nucleotide is 2′-deoxyribonucleotide (DNA) and when at least four 3′-terminal bases of NA are DNA bases.

Usually, the NA component contains the major heterocyclic bases naturally found in nucleic acids (uracil, cytosine, thymine, adenine and guanine). In some embodiments, the NA contains nucleotides with modified, synthetic or unnatural bases, incorporated individually or multiply, alone or in combination. Preferably, modified bases increase thermal stability of the probe-target duplex in comparison with probes comprised of only natural bases (i.e., increase the hybridization melting temperature of the probe duplexed with a target sequence). Modified bases include naturally-occurring and synthetic modifications and analogues of the major bases such as, for example, hypoxanthine, 2-aminoadenine, 2-thiouracil, 2-thiothymine, inosine, 5-N⁴-ethenocytosine, 4-aminopyrrazolo[3,4-d]pyrimidine and 6-amino-4-hydroxy-[3,4-d]pyrimidine. Any modified nucleotide or nucleotide analogue compatible with hybridization of a hyperthermostable endonuclease IV substrate probe with a target nucleic acid conjugate to a target sequence is useful in the practice of the invention, even if the modified nucleotide or nucleotide analogue itself does not participate in base-pairing, or has altered base-pairing properties compared to naturally-occurring nucleotides. Examples of modified bases are disclosed in U.S. Pat. Nos. 5,824,796; 6,127,121; 5,912,340; and PCT Publications WO 01/38584; WO 01/64958, each of which is hereby incorporated herein by reference in its entirety. Preferred modified bases include 5-hydroxybutynyl uridine for uridine; 4-(4,6-Diamino-¹H-pyrazolo[3,4-d]pyrimidin-3-yl)-but-3-yn-1-ol, 4-amino-¹H-pyrazolo[3,4-d]pyrimidine, and 4-amino-¹H-pyrazolo[3,4-d]pyrimidine for adenine; 5-(4-Hydroxy-but-1-ynyl)-1H-pyrimidine-2,4-dione for thymine; and 6-amino-¹H-pyrazolo[3,4-d]pyrimidin-4(5H)-one for guanine. Particularly preferred modified bases are “Super A®,” “Super G®: 4-hydroxy-6-amino pyrazolopyrimidine” (www.elitechgroup.com) and “Super T®”. Modified bases preferably support the geometry of a naturally occurring B-DNA duplex. Modified bases can be incorporated into any position or positions in a hyperthermostable endonuclease IV substrate probe, but preferably are not incorporated as the 3′-terminal base.

In some instances a minor groove binder can be attached to NA. Minor groove binders have be disclosed in U.S. Pat. No. 5,801,155 and U.S. Pat. No. 6,312,894 which are both incorporated by reference. A preferred minor groove binder is DPI₃.

In another embodiment, some or all nucleotides of NA are substituted or contain independently different sugar-phosphate backbone modifications including 2′-O-alkyl RNA nucleotides, phosphorotioate internucleotide linkage, methylphosphonate, sulfamate (e.g., U.S. Pat. No. 5,470,967) and polyamide (i.e., peptide nucleic acids, PNA), LNA (locked nucleic acid), and the like. Such modifications and others of potential use in the present invention are described, for example, in Boutorine, et al., Biochimie 76:23 (1994); Agrawal, et al., Proc. Natl. Acad. Sci. 88:7595 (1991); Mag, et al., Nucleic Acids Res. 19:1437 (1991); Kurreck, Eur. J. Biochem. 270:1628 (2003); Lesnik, et al., Biochemistry 32:7832 (1993); Sproat, et al., Nucleic Acids Symp. Ser. 24:59 (1991); Iribarren, et al., Proc. Natl. Acad. Sci. 87:7747 (1990); Demidov, Trends Biotechnol. 21:4 (2003); Nielsen, Methods Mol. Biol. 208:3 (2002); Nielsen and Egholm, Curr. Issues Mol. Biol. 1:89 (1999); Micklefield, Curr. Med. Chem. 8:1157 (2001); Braasch, et al., Chem. Biol. 8:1 (2001); and Nielsen, Curr. Opin. Biotechnol. 12:16 (2001).

In another embodiment, some or all nucleotides of NA are substituted with a quencher and fluorophore pair. There is extensive guidance in the art for selecting quencher and fluorophore pairs and their attachment to oligonucleotides (Haugland, R. P., HANDBOOK OF FLUORESCENT PROBES AND RESEARCH CHEMICALS, Sixth Edition, Molecular Probes, Eugene, Oreg., 1996; U.S. Pat. Nos. 3,996,345 and 4,351,760 and the like). Preferred quenchers are described in co-owned U.S. Pat. No. 6,727,356 and U.S. Pat. No. 6,790,945 incorporated herein by reference, and dyes from Biosearch Technologies, Inc. (provided as Black Hole™ Quenchers: BH-1, BH-2 and BH-3), Dabcyl, TAMRA and carboxytetramethyl rhodamine. The terms “fluorescent label” or “fluorophore” refers to compounds with a fluorescent emission maximum between about 400 and 900 nm. These compounds include, with their emission maxima in nm in brackets, Cy2™ (506), GFP (Red Shifted) (507), YO-PRO™-1 (509), YOYO™-1 (509), Calcein (517), FITC (518), Fluor X™ (519), Alexa™ (520), Rhodamine 110 (520), 5-FAM. (522), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO®-1 (533), JOE (548), BODIPY® 530/550 (550), Dil (565), BODIPY® TMR (568), BODIPY® 558/568 (568), BODIPY® 564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red® (615), Nile Red (628), YO-PRO™-3 (631), YOYO™-3 (631), R-phycocyanin (642), C-Phycocyanin (648), TO-PRO™-3 (660), TOTO®-3 (660), DiD DilC(5) (665), Cy5™ (670), Thiadicarbocyanine (671), Cy5.5 (694). Fluorophores further refers to fluorescent derivatives disclosed in WO 03/023357, and U.S. application Ser. No. 11/202,635, U.S. application Ser. Nos. 11/360,040 and 12/244,712.

Within the scope of present invention, modifications of the bases and sugar-phosphate backbone as well as other functional moieties conjugated with the probe can serve to improve the sequence specificity of the target-probe duplex formation. In particular, binding between the probe and a matched target nucleic acid is detectably increased over binding to a mismatched target nucleic acid. By “matched target nucleic acid” is intended a target nucleic acid that contains a sequence that is completely complimentary to the probe sequence. By “mismatched target nucleic acid” is intended a polynucleotide that contains a sequence that is partially complimentary to the probe sequence such that it contains at least one mismatched, non-complimentary base, deletion or insertion in comparison to the probe sequence. For example, use of modified bases in an endonuclease IV substrate probe allows for more stable base pairs than when using natural bases and enables the use of shorter probes for the same reaction conditions. Reduction of the probe length increases the ability of the probe to discriminate a target polymorphism as small as a Single Nucleotide Polymorphism (“SNP”) due to a proportional increase in the contribution of each duplex base pair to the overall duplex stability. In general, the shorter the probe, the greater the relative contribution of an individual base pair in to the overall duplex stability, and the better the probe discrimination of the target polynucleotide polymorphism.

Linker (“L”) Component of the Endonuclease IV Substrate Probe

A linker L may be present between the oligonucleotide sequence NA and the functional, chemical tail R or R′. In one embodiment a phosphoramidite linker 35 was synthesized as shown in Reaction Scheme 1, below.

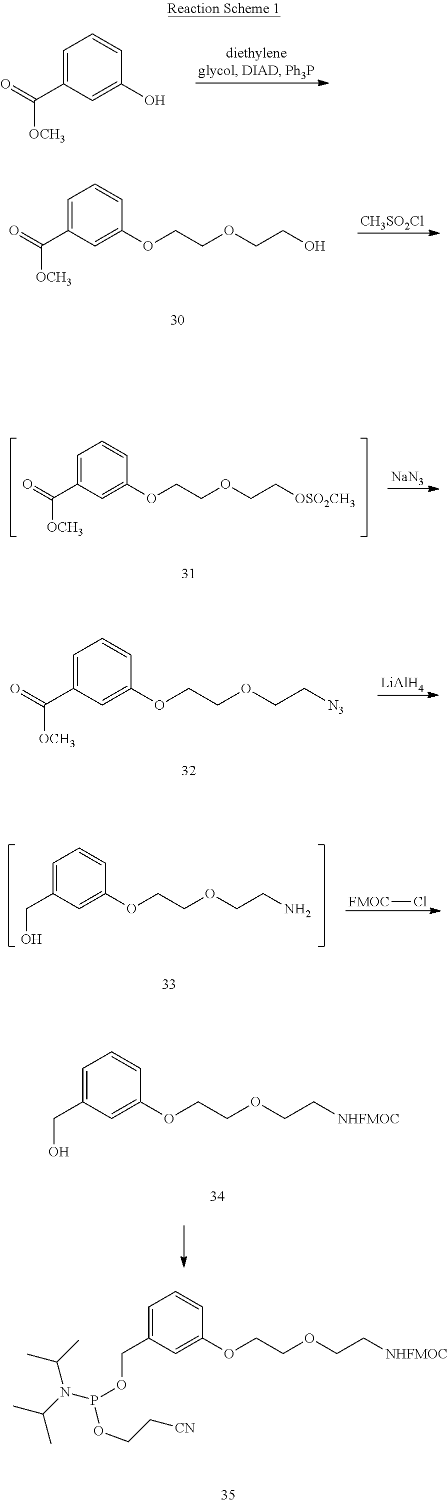

As shown in Reaction Scheme 1, methyl 3-hydroxybenzoate may be reacted with diisopropyazodicarboxylate and triphenyl phosphine to yield methyl 3-{2-[2-ethoxy]ethoxy}benzoate 30. Compound 30 may be treated with p-toluene sulfonyl chloride to yield the crude tosylate 31 which may be reacted without purification with sodium azide to give the desired azide 32. Azide (32) may be reduced with LiAlH₄ to yield the aminoalcohol 33 which can be converted directly to the N-Fmoc 34 by reaction with 9-fluorenylmethyl chloroformate. The N-Fmoc derivative may be reacted with 2-cyanoethyl N,N,N′N′-tetraisopropylphosphordiamidite to convert to the desired phosphoramidite 35.

In another embodiment a phosphoramidite linker 44 was synthesized as shown in Reaction Scheme 2.

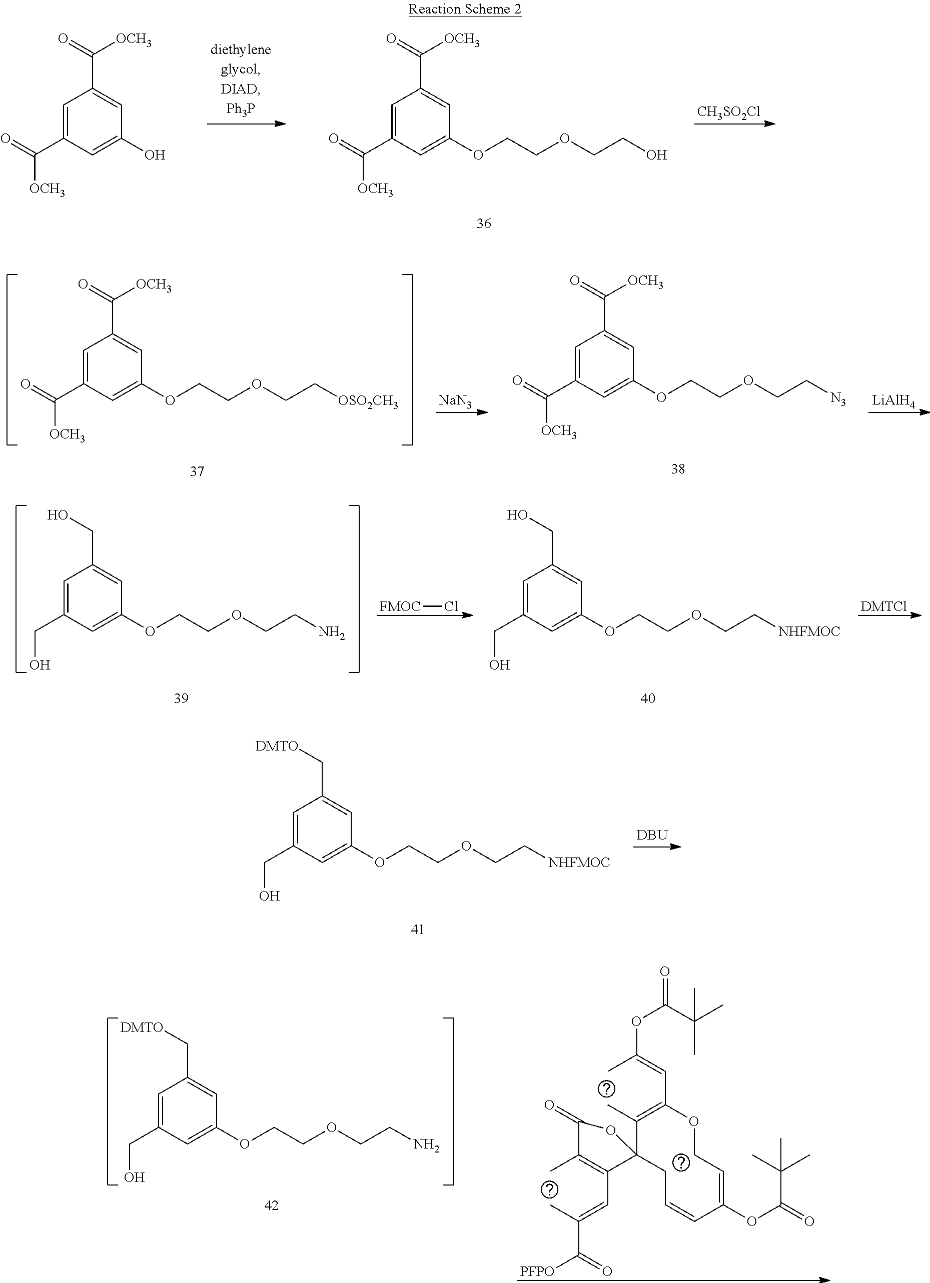

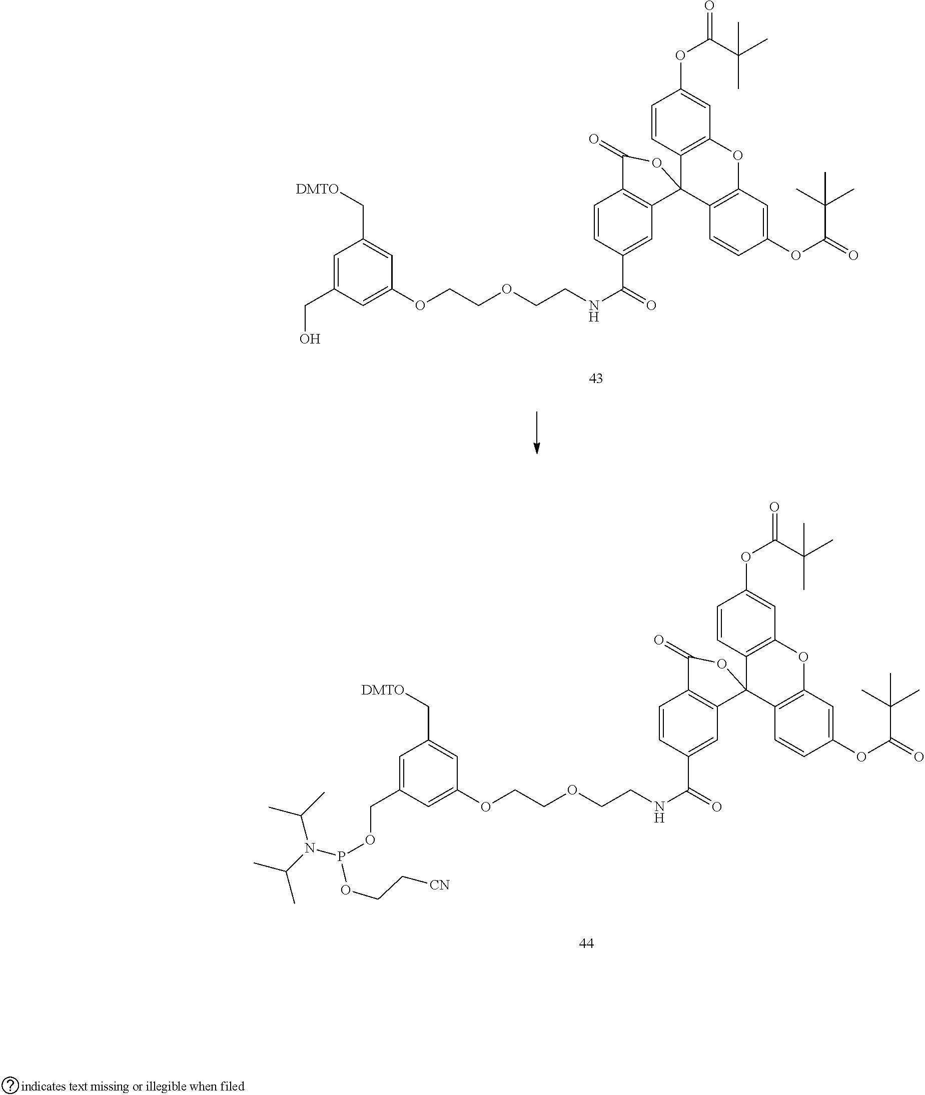

As shown in Reaction Scheme 2, dimethyl 5-hydroxyisophthalate may be reacted with triphenylphosphine in the presence of diisopropylazodicarboxylate to yield methyl 5-{2-[2-ethoxy]ethoxy}-3-(methoxycarbonyl)benzoate (36). Compound 36 may be treated with p-toluene sulfonyl chloride to yield the crude tosylate 37 which may be reacted without purification with sodium azide to give the desired azide 38. Azide 38 may be reduced with LiAlH₄ to yield the aminoalcohol 39 which is converted directly to the N-Fmoc 40 by reaction with 9-fluorenylmethyl chloroformate. The N-Fmoc derivative 40 may be reacted with dimethoxytrityl chloride to give the mono-DMT substituted diol 41. This may be treated with DBU to yield the amine 42 which may be directly reacted with pentafluorophenyl dipivaloylfluorescein-6-carboxylate (29) to afford the mono-DMT substituted fluorescein 43, which may be reacted with 2-cyanoethyl N,N,N′N′-tetraisopropylphosphordiamidite to convert to the desired phosphoramidite 35.

L is a linker that may include linear or acyclic portions, cyclic portions, aromatic rings or combinations thereof each of which contain from 0-3 of any of N, O, P or S. Preferred linker compositions allow less than 5% non-specific cleavage by the hyperthermostable endonuclease IV enzyme in the no-template control, more preferred compositions allow less than 2.5% and 1% non-specific cleavage. A variety of linking groups and methods are known to those of skill in the art for attaching fluorophores, quenchers and minor groove binders to the 5′ or 3′ termini of oligonucleotides. See, for example, Eckstein, editor, OLIGONUCLEOTIDES AND ANALOGUES: A PRACTICAL APPROACH (IRL Press, Oxford, 1991); Zuckerman et al., Nucleic Acids Research, 15:5305-5321 (1987); Sharma et al., Nucleic Acids Research, 19:3019 (1991); Giusti et al., PCR Methods and Applications, 2:223-227 (1993), Fung et al., U.S. Pat. No. 4,757,141; Stabinsky, U.S. Pat. No. 4,739,044; Agrawal et al., Tetrahedron Letters, 31:1543-1546 (1990); Sproat et al., Nucleic Acids Research, 15:4837 (1987); Nelson et al., Nucleic Acids Research, 17:7187-7194 (1989); and the like. Still other commercially available linking groups can be used that can be attached to an oligonucleotide during synthesis, e.g., available from Glen Research (www.glenresearch.com.) and TriLink (www.trilinkbiotech.com) Other methodologies for attaching a fluorophore to an oligonucleotide portion involve the use of phosphoramidite chemistry at the conclusion of solid phase synthesis by way of dyes derivatized with a phosphoramidite moiety. See, for example, Woo et al., U.S. Pat. No. 5,231,191; Hobbs, Jr., U.S. Pat. No. 4,997,928; Reed, et al., PCT publication No. WO 01/42505; U.S. Pat. No. 6,653,473 and U.S. application Ser. No. 10/026,374.

A series of novel endonuclease IV substrate probes containing cleavage sites is disclosed in Table 2. In these probes, F1 is a detectable label, including fluorophores such as Gig Harbor Green and FAM. Surprisingly, the cleavable substrate disclosed in U.S. Pat. No. 7,252,940, which can be cleaved by E. coli Endonuclease IV (Compound 1 in Table 2), is not cleaved by Tth Endonuclease IV (New England Biolabs, Ipswitch, Mass.), isolated from a hyperthermostable bacteria. It appears that the hyperthermostable Tth Endonuclease IV requires a more flexible cleavable linker compared the E. coli endonuclease IV. Three different cleavable substrate types 3, 8, and 14, gave particularly high signal to background ratios when used in assays with Tth Endonculease IV.

TABLE 2
The effect of the linker L in the probe (Q-TCCGTA*TGGTG-L-F1) specific for M. tuberculosis on
the signal/background ratio (S/B) in the hyperthermostable Tth Endonuclease IV assay, in an embodiment of
the invention. S is signal in relative fluorescent units. F1 is Gig Harbor Green for probes 1-7 and FAM for
probes 8-14.

| | | | S/B |
|---|---|---|---|
| Probe # | Probe Containing Linker L | S | Ratio |
| 1 | 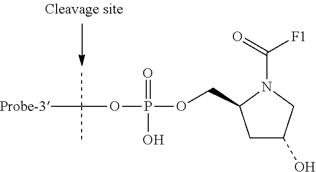 | 0 | No cleavage |
| 2 | 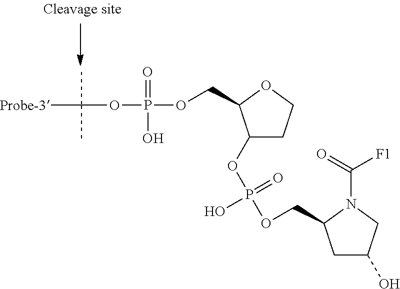 | 0 | No cleavage |
| 3 | 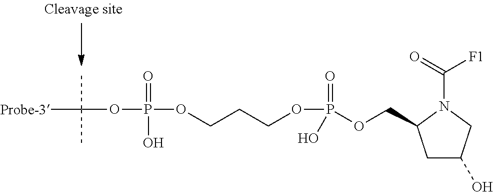 | 6.8 | 680 |
| 4 | 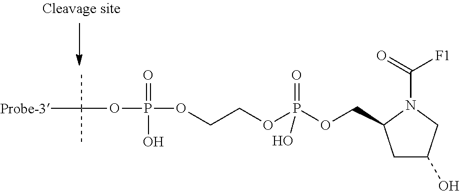 | 6.1 | 61 |
| 5 | 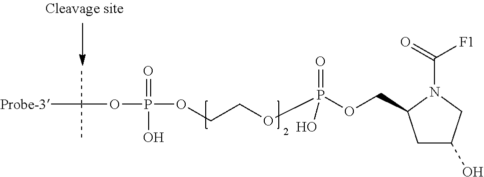 | 12.3 | 83 |
| 6 | 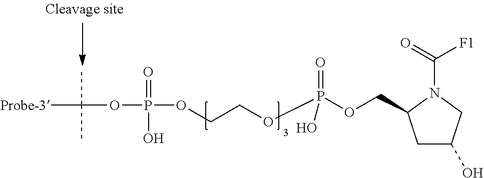 | 16.5 | 110 |
| 7 | 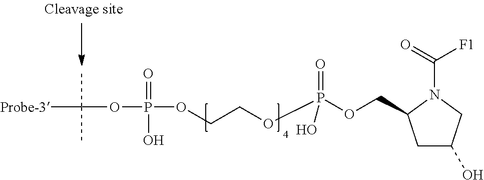 | 19 | 211 |
| 8 | 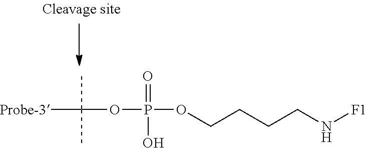 | 43 | 2166 |
| 9 | 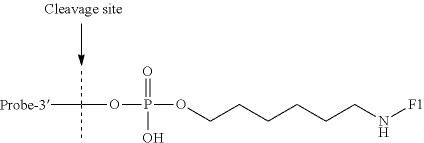 | 70 | 68 |
| 10 | 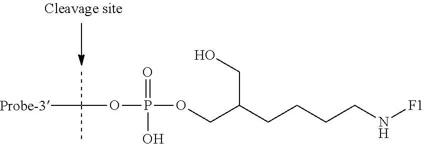 | 18 | 180 |
| 11 | 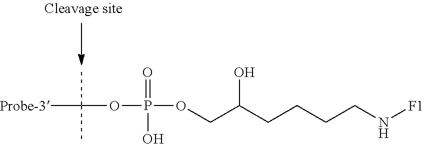 | 8.5 | 71 |
| 12 | 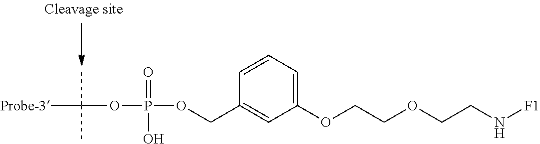 | 32 | 158 |
| 13 | 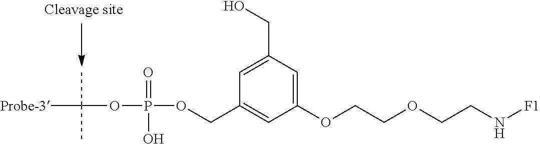 | 7.6 | 76 |
| 14 | 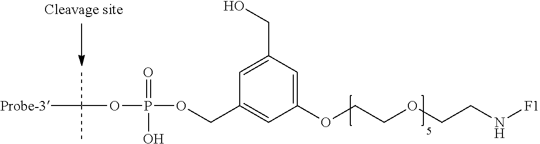 | 13.4 | 1340 |

In one embodiment, the endonuclease IV substrate has the following structure:

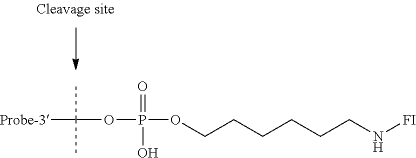

In one embodiment, the endonuclease IV substrate has the following structure:

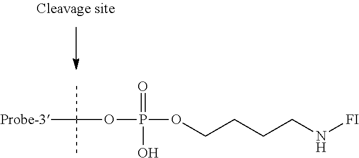

In one embodiment, the endonuclease IV substrate has the following structure:

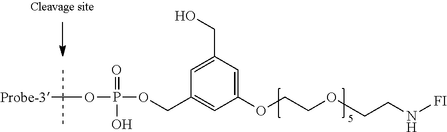

In the above embodiments, F1 is a detectable reporter group. In one additional embodiment, the endonuclease IV substrate has a signal/background ratio of greater than 100. In another embodiment the hyperthermostable endonuclease IV substrate probe has a signal/background ratio of greater than 50.

Functional Tail (“R” or “R”) Component of the Endonuclease IV Substrate Probe

The functional tail R or R′ may enable detection of a thermophilic or hyperthermophilic cleavage reaction. The structure of R or R′ can be of any size and composition as long as the linker L supports the template-specific, hyperthermostable endonuclease IV tail-cleavage reaction. R or R′ can be as large as a natural protein with molecular mass up to 1,000,000 Daltons or it can be as small as a single atom (i.e., a radioactive isotope, such as hydrogen or iodine). Since the enzymatic hydrolysis occurs between the 3′-terminal oxygen atom of the oligonucleotide sequence NA and the phosphorus atom of the phosphodiester bond, for the purposes of the present invention, the phosphate moiety of the endonuclease IV substrate probe is considered a part of the functional tail LR'. For example, when R′ is hydrogen (R′═H), the functional tail of the probe is a L-phosphate moiety —P(O)(OH)(OL) or —PO₂(OL)⁻. The tail R′ may be hydrophobic or hydrophilic, electrically neutral, positively or negatively charged. It may be comprised of or include independently different functional groups, including mass tags, nanoparticles, fluorescent or non-fluorescent dyes, linkers, radioisotopes, functional ligands like biotin, oligopeptides, carbohydrates and the like. For example, as demonstrated herein, the hyperthermostable endonuclease IV from Thermus Thermophilus Endonuclease IV (Tth Endonuclease IV) efficiently cleaves from the 3′-end of a probe bound to the target nucleic acid a relatively hydrophilic, negatively charged fluorescein moiety as well as an electrically neutral, hydrophobic quenching dye.

The tail R or R′ can contain components that improve specificity by blocking non-specific cleavage reactions in the absence of a target molecule without affecting the target-dependent, specific reaction. More specifically, cleavage specificity and efficiency is primarily determined by the linker L. It is within the scope of present invention that the tail R or R′ or some structural components of it may improve the specificity of the target-probe or enhancer-probe complementary binding so that the thermodynamic difference in the probe binding to matched and mismatched target nucleic acids is increased. Examples of such structural components are minor groove binders (MGBs).

### III HYPERTHERMOSTABLE ENZYME

In one embodiment of the invention, the hyperthermostable endonuclease IV is either a native or recombinant hyperthermostable endonuclease IV isolated from a hyperthermophile listed in Table 1.

In one embodiment of the invention, the hyperthermostable endonuclease IV is isolated from Thermos Thermophilus.

In one embodiment of the invention, the hyperthermostable endonuclease IV is an engineered enzyme. In another embodiment, the hyperthermostable endonuclease IV has a thermal stability of >80° C. In another embodiment the hyperthermostable endonuclease has a thermal stability of between 80° C. and 110° C.

In one embodiment the hyperthermostable endonuclease IV requires the presence of a metal ion. Examples of metal ions include Mg²⁺, Co²⁺, Mn²⁺, Ca²⁺, and Zn²⁺.

In another embodiment the hyperthermostable endonuclease requires the presence of a detergent for optimal activity. Detergents may include any ionic, anionic, nonionic, cationic, or ampholytic detergent or surfactant. For example, 1-heptanesulfonic acid, 1-octanesulfonic acid, benzethonium hydroxide, Brij® (Polyethylene glycol dodecyl ether) 30, Brij® 35, CHAPS (3-[(3-Cholamidopropyl)dimethylammonio]-1-propanesulfonate), cholic acid, decaethylene glycol monododecyl ether, digitonin, docusate sodium, hexadecylpyridinium chloride monohydrate, hexadecyltrimethylammonium bromide, IGEPAL® CA-210(Polyoxyethylene (2) isooctylphenyl ether), IGEPAL® CA-520 (Polyoxyethylene (5) isooctylphenyl ether), IGEPAL® CA-630 (Octylphenoxy)polyethoxyethanol, IGEPAL® CA-720 (Polyoxyethylene (12) isooctylphenyl ether), lithium dodecyl sulfate, N-dodecyl-N,N-dimethyl-3-ammonio-1-propanesulfonate, N-lauroylsarcosine, octyl β-D-glucopyranoside, poly(ethylene glycol), polyoxyethylene (20) sorbitan monolaurate, polysorbat 60, polysorbate 20, polysorbate 80, saponin, sodium 1-decanesulfonate, sodium 1-heptanesulfonate, sodium cholate hydrate, sodium deoxycholate, sodium dodecyl sulfate, sodium glycochenodeoxycholate, sodium glycocholate hydrate, sodium hexanesulfonate, sodium octyl sulfate, sodium pentanesulfonate, sodium taurodeoxycholate hydrate, sodium thiosulfate, TWEEN® 20 (Polyethylene glycol sorbitan monolaurate), TWEEN® 21 (Polyoxyethylenesorbitan monolaurate), TWEEN® 80 (Polyethylene glycol sorbitan monooleate), taurocholic acid, tetramethylammonium hydroxide pentahydrate, Triton® (4-(1,1,3,3-Tetramethylbutyl)phenyl-polyethylene glycol) X-100, Triton® X-102, Triton® X-114, Triton® X-15, Triton® X-165, Triton® X-305, Triton® X-405, tyloxapol, n-dodecyl β-D-maltoside, and combinations thereof.

### IV METHOD OF DETECTING A TARGET NUCLEIC ACID IN A SAMPLE

The invention further comprises a method of detecting a target nucleic acid in a sample. In an embodiment of the invention, the method comprises the steps of: a) contacting the sample with at least one endonuclease IV substrate probe, as described herein, and a hyperthermostable endonuclease 1V, such that the endonuclease IV substrate probe hybridizes to the target nucleic acid to form a reaction mixture; b) incubating the reaction mixture under reaction conditions sufficient to allow said hyperthermostable endonuclease VI to cleave the phosphodiester bond attaching the functional tail R to the 3′ terminal of the oligonucleotide sequence NA, wherein the hyperthermostable endonuclease VI preferentially cleaves the phosphodiester bond attaching the functional tail R to the oligonucleotide sequence NA when the oligonucleotide sequence NA is hybridized with a complementary target nucleic acid sequence in comparison to when the oligonucleotide sequence NA is unhybridized or hybridized to a non-complementary target nucleic acid; and c) detecting the reporter group on the cleaved functional tail R, whereby the target nucleic acid is detected.

### V DETECTION OF NUCLEIC ACID POLYMORPHISM

hyperthermostable endonuclease VI substrate probes are particularly suited for DNA genotyping or detection of two related target nucleic acids that share essentially the same sequence and that are different by a number of bases within the sequence of interest. Most commonly, the difference in the target DNA sequences of interest are as small as one base (SNP). AP endonucleases, such as hyperthermostable endonuclease IV, generally bind to the DNA on either side from an abasic site and are affected by mismatched base pairs residing in proximity to their preferred enzyme binding site. A mismatched base pair that resides within the region recognized by the endonuclease IV substrate probe has a negative effect on the enzyme-DNA-substrate binding, and consequently impedes the catalytic rate of tail-cleavage, as measured by a detectable reporter group signal. AP endonucleases identify mismatched base pairs located in the region of their binding sites by preferentially cleaving the functional tails R of a hyperthermostable endonuclease IV substrate probe duplexed with a target nucleic acid sequence having matched base pairs located outside the enzyme binding region in comparison to cleaving the tail R of a probe duplexed with a target nucleic acid having mismatched base pairs in the enzyme binding region.

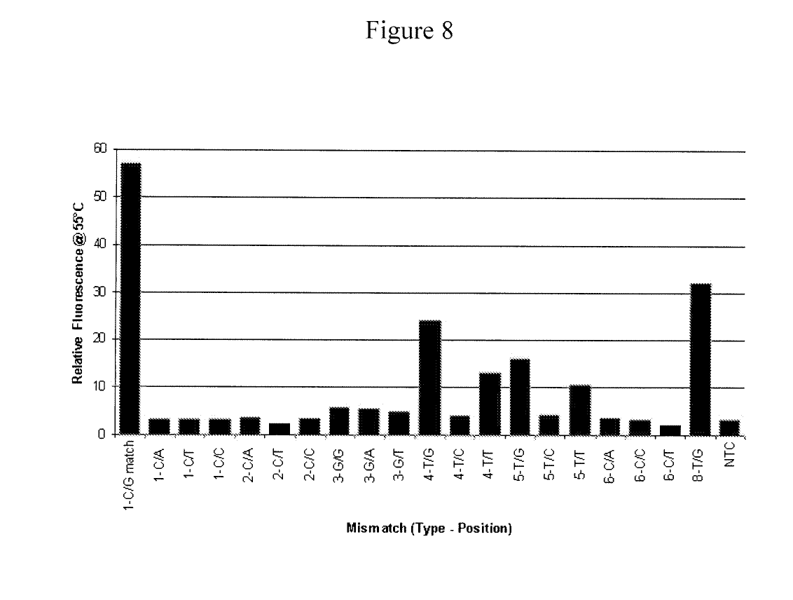

Hyperthermostable endonuclease VI substrate probes find particular use in detecting base pair mismatches that potentially exist at a known or suspected location in a target nucleic acid. Usually in such assays, two or more different hyperthermostable endonuclease VI substrate probes are contacted with one or more target nucleic acids in a sample, each probe having a nucleic acid sequence differing at one or more bases and distinctly detectable reporter groups. FIG. 8 shows a comparison of the change in relative signal fluorescence of match and different mismatches at different positions in a 14-mer probe in an Endo IV assay run at 55° C. Preferably, the mismatch is positioned within 8 nucleotides from the 3′ end of the probe, more preferably at the 7, 6, 5, 4 or 3 position from the 3′ end of the probe, and most preferably at the 1 or 2 position from the 3′ end of the probe, where position 1 is the 3′ end nucleotide. In a most preferred embodiment the mismatch is located at position 2 from the 3′ end of the probe. Base pair mismatch identification assays using a hyperthermostable endonuclease IV substrate probe can be conveniently carried out in combination with amplification systems, particularly with isothermal amplification systems.

In one embodiment of the hyperthermostable endonuclease IV substrate probe the mismatch is located in any of positions 1 to 8 from the 3′-end of the hyperthermostable endonuclease IV cleavable substrate, with linker 8. In a preferred embodiments the mismatch is located in positions 1-2 and 1-4.

In one embodiment the mismatch is located in any of positions 1 to 8 from the 3′-end of the hyperthermostable enzyme cleavable substrate, with linker 3. In a preferred embodiments the mismatch is located in positions 1-2 and 1-4.

In one embodiment the mismatch is located in any of positions 1 to 8 from the 3′-end of the hyperthermostable enzyme cleavable substrate, with linker 14. In a preferred embodiments the mismatch is located in positions 1-2 and 1-4.

### Example 1

5′-Q-oligonucleotide-L-F1 probes of the structures 1-7 (Table 2) were synthesized using a DNA synthesizer starting from the solid support 15 (FIG. 6) (Kutyavin, I. V., 2006) followed by one of the spacer phosphoramidites 16-21 (FIG. 6), then followed by 3′-DNA phosphoramidites to incorporate probe sequence, and, finally, by the Epoch Eclipse® Quencher phosphoramidite (Glen Research Corp.). Spacer phosphoramidites 16, 17 and 20 (FIG. 6) were purchased from Glen Research Corp. Spacer phosphoramidites 18 and 19 (FIG. 6) were prepared as described in EP 1136569. Spacer phosphoramidite 21 (FIG. 6) was prepared as described in U.S. Pat. No. 5,574,142.

Probes of the structures 8-14 (Table 2) were synthesized starting from the Epoch Eclipse® Quencher solid support (Glen Research Corp.) followed by 5′-DNA phosphoramidites to incorporate the probe sequence and, finally, by one of the linker phosphoramidites 22-28 (FIG. 7). Linker phosphoramidite 22 (FIG. 7) was prepared as described by U.S. Pat. No. 5,925,744. Phosphoramidites 23 and 24 (FIG. 7) were purchased from Glen Research Corp. Linker phosphoramidite 25 (FIG. 7) was prepared as described by Nelson et al., Nucleosides and Nucleotides, 1951-1959). Phosphoramidite 28 (FIG. 7) was prepared as described in U.S. Pat. No. 7,381,818.

### Example 2

Fluorescein was incorporated post-synthetically into the probes 8, 11 and 12 (Table 2). In the case of linker phosphoramidites 22, 25 and 26 (FIG. 7) the fluorophor was incorporated post-synthetically (Reaction Scheme 3) using PFP bis-pivaloylfluorescein-6-carboxylate (29) (Reaction Scheme 3) prepared as described by Jadhav et al., 1997).

Briefly, an amine-tailed probe precursor (˜100 nmoles, triethylammonium salt) was dissolved in 80 μl of DMSO and treated with 2 ml of triethylamine and 1 mg of 29 (Reaction Scheme 3). After being kept at room temperature for 5 hrs the reaction was diluted with a 2% solution of NaClO₄ in acetone (1.5 ml). Precipitated material was collected by centrifugation, washed with acetone (1 ml) and dried. Crude conjugate was purified by C18 HPLC (4.6×250 mm, C18 Luna, 10 μm, Phenominex) using a gradient of acetonitryl in 0.1 M triethylammonium bicarbonate buffer. Purified conjugate was dried in a SpeedVac concentrator and treated with concentrated NH₄OH at 70° C. for 2 hrs to remove the pivaloyl protection. The final conjugates 8, 11 and 12 (Table 2) were purified by C18 HPLC and dried as described above.

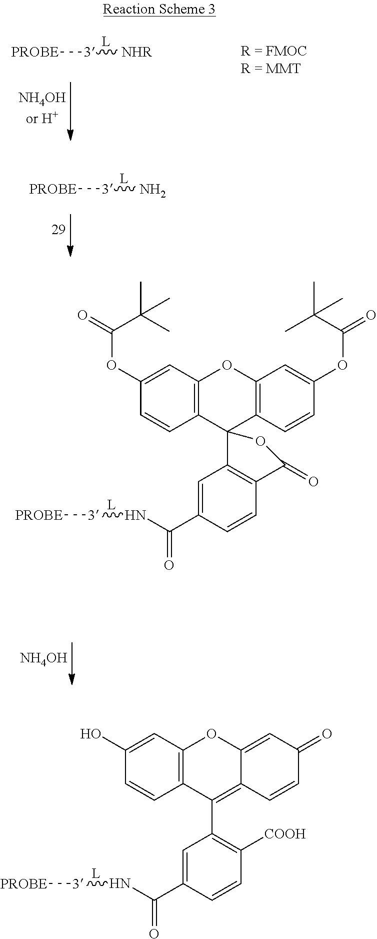

### Example 3

This example illustrates the dependence of cleavage specificity and efficiency in a PCR/Thermostable Endonuclese IV assay. A probe specific for Mycobacterium tuberculosis was designed with the following sequence Q-TCCGTA*TGGTG-L-F1, where Q is the Eclipse Dark Quencher, F1 is the Gig Harbor Green Dye and A* is the Super A. A series of this probe was synthesized with different linkers L shown in Table 2. This table also shows the signal/background ratios for each oligonucleotide when evaluated with the hyperthermostable Tth Endonuclease IV (generously donated by New England Biolabs, Inc; www.neb.com).

Surprisingly probe 1 (Table 2, U.S. Pat. No. 7,252,940) containing the linker that was cleaved specifically by Escherichia coli Endonuclease IV, was not cleaved at all by the hyperthermostable Tth Endocnuclease IV. The linker in probe 2 (Table 2) was also not cleaved while the linker in probe 5 (Table 2) was cleaved non-specifically by the Tth endonuclease IV. Specific cleavage was observed with all the other probes in Table 2 with signal/background (S/N) ratios raging from about 6 to more than 2000. The linker in probe 8 (Table 2) gave the highest S/N ratio.

### Example 4

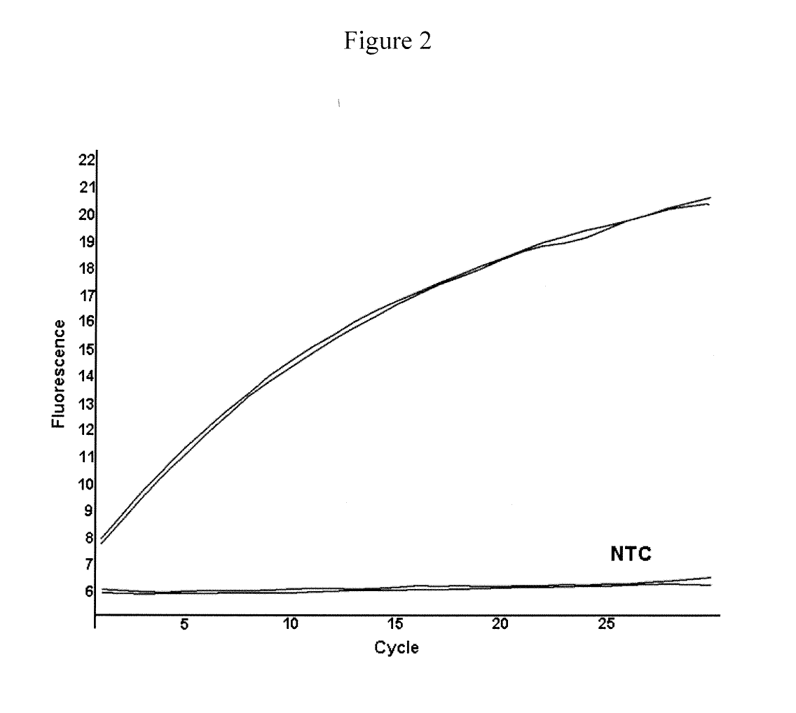

This example illustrates a closed tube PCR amplification followed by post PCR hyperthermostable endonuclease IV detection of 1 ng M. tuberculosis with the probe containing linker 6 (linker shown in Table 2, amplification shown in FIG. 2). A closed tube assay contains PCR buffer with 400 μM. ZnCl₂, 100 nM forward primer, 1000 nM reverse primer, JumpStart polymerase (Sigma), 500 nM probe, 0.02 U Tth Endo IV and 1 ng of M. tuberculosis genomic DNA. Fifty cycles of three-step PCR profile (95° C. for 5 s, 58° C. for 30 s, 72° C. for 30 s) were run after an initial 2 min denaturation step at 95° C. followed by post PCR isothermal at 50° C. for 60 minutes with the Tth Endonuclease IV reaction.

### Example 5

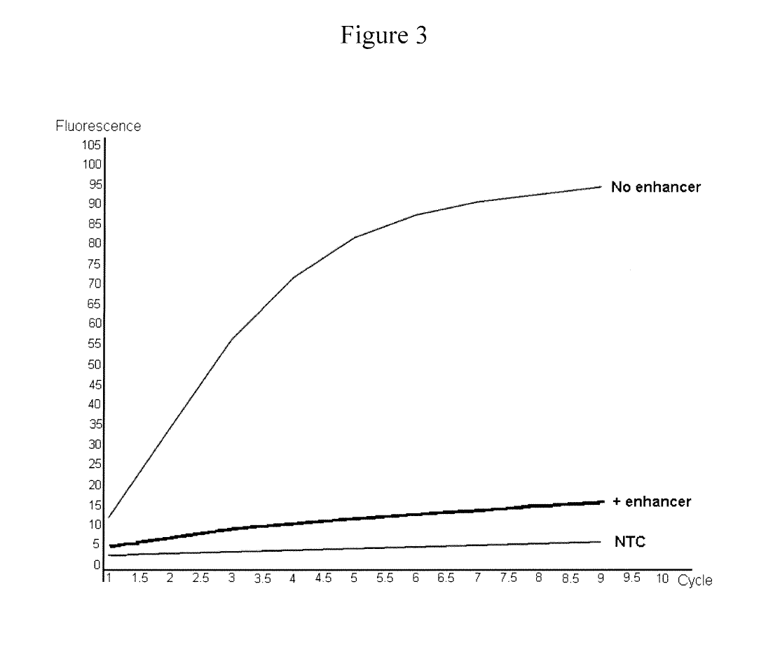

This example illustrates the inhibition of the Tth Endonuclease cleavage by enhancer. The probe Q-TCCGTA*TGGTG-L-GG containing linker 6 (Table 2), was separated by one base at the 3′-end of the probe by the enhancer ATAA*CGT*CTTTCA*. A* and T* represent respectively the base Super A® and Super T®, used to increase the stability of the probe and enhancer. Cleavage was investigated with a complementary synthetic target. The results shown in FIG. 3, indicated significant inhibition of the Tth Endonuclease IV cleavage by the presence of the enhancer.

### Example 6

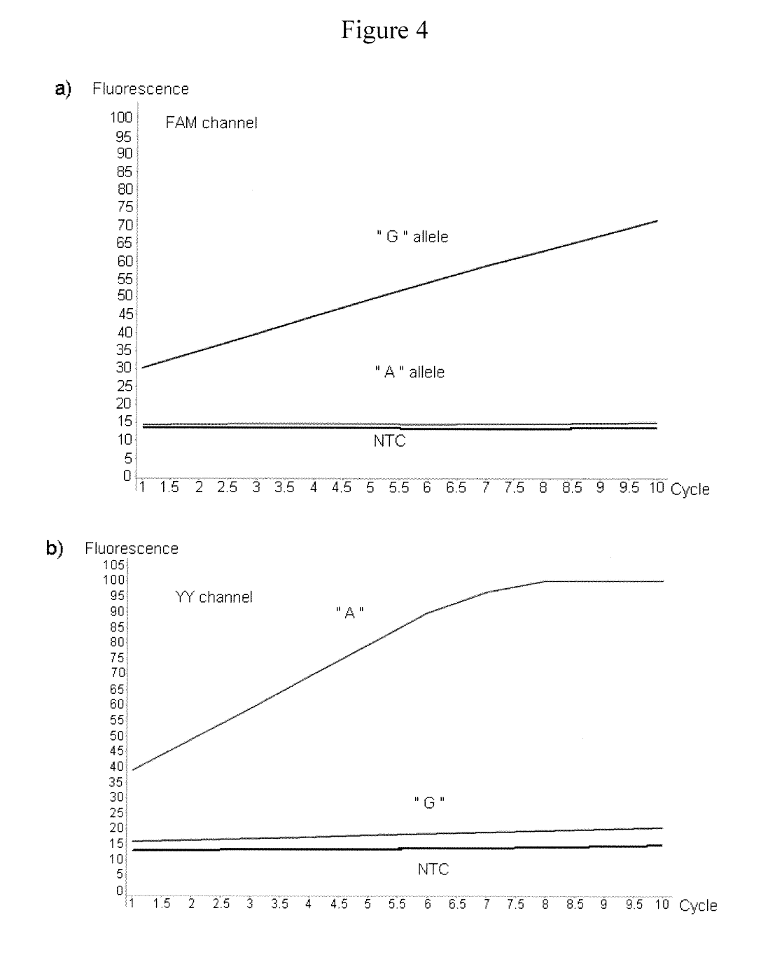

This example illustrates single nucleotide polymorphism (SNP) detection in the closed tube format. Probes were designed to detect in G/A polymorphism in wild type and mutant PCR synthetic templates. The closed tube procedure of Example 3 was used with the following modifications: post PCR isothermal was performed at 45° C. for 20 minutes with the Tth Endonuclease IV enzyme. The probe for the wild type allele is Q-TACCTT*CTTCG-L-GG and for the mutant is Q-TACCTT*CTTTG-L-YY. T* is Super T® and the alleles are shown in bold and is positioned in the second base from the 3′-end of the two probes, respectively. Gig Harbor Green as similar excitation and emission fluorescent properties than FAM. As shown in FIG. 4, excellent specific detection of the alleles is obtained.

### Example 7

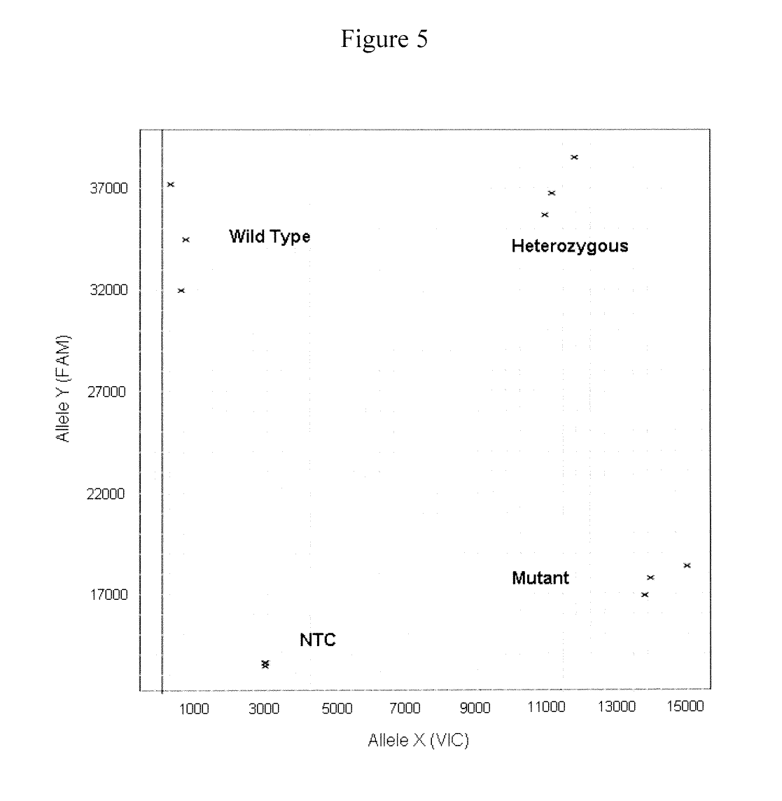

This example illustrates the excellent SNP detection can also be obtained with the probes described in Example 5, when results are presented in a scatter plot (FIG. 5). The reaction conditions were the same as those described in Example 3, except that the Tth Endonuclease IV concentration was lowered to 0.025U, post PCR detection was performed at 45° C. and each probe was at 700 mM concentration using 20 two step cycles (45° C. for 60s and 75° C. for 1 second).

### Example 8

This examples illustrates change in relative signal fluorescence of match and different mismatches at different positions in a 14-mer probe in an Endo IV assay run at 55° C. (FIG. 8). The probe sequence of the matched probe and target sequence are, respectively, 5′-Q-ACTCGGTCCTTGCC-FL-3′ and 5′-AGTCACAGTCGGTGCCAATGTGGCGGGCAAGGACCGAGTCG-3′. NTC is the no template control. The probe sequences are shown in Kutyavin et al (2006).

### Example 9

### Synthesis of Phosphoramidites 35 and 44 (Reaction Schemes 1 and 2)

### Methyl 3-{2-[2-ethoxy]ethoxy}benzoate (30)

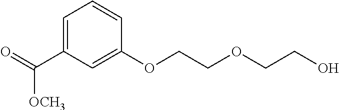

Diisopropylazodicarboxylate (8.3 ml, 42.1 mmol) was added over 3 min to a stirred solution of methyl 3-hydroxybenzoate (5.0 g, 32.9 mmol), diethylene glycol (10 ml, 105 mmol) and triphenylphosphine (11.2 g, 42.7 mmol) in 50 ml of anhydrous THF. The reaction was stirred for 2 h and then concentrated. The resulting residue was suspended in ˜75 ml of ethyl ether and cooled to 0° C. Precipitated solids were removed by filtration and the filtrate was concentrated to a viscous liquid, which was then chromatographed on silica eluting with hexane/ethyl acetate. Concentration of the pure product fractions and drying under vacuum afforded 5.0 g (63%) of the title compound as a viscous oil. ¹H NMR (DMSO-d6) δ 7.54 (ddd, J₁=7.8 Hz, J₂=1.2 Hz, J₃=1.2 Hz, 1H), 7.44 (m, 2H), 7.24 (ddd, J₁=8.4 Hz, J₂=2.7 Hz, J₃=1 Hz, 1H), 4.63 (t, J=5.5 Hz, 1H), 4.15 (m, 2H), 3.85 (s, 3H), 3.76 (m, 2H), 3.51 (m, 2H), 3.50 (s, 2H).

### Methyl 3-{2-(2-azidoethoxy)ethoxy}benzoate (32)

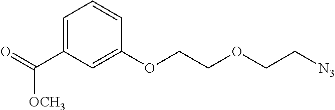

p-Toluene sulfonyl chloride (4.76 g, 25 mmol) was added in one portion to a stirred, cold (ice/water bath) solution of 30 (5.0 g, 20.8 mmol) and triethylamine (4.35 ml, 31 mmol) in 50 ml of anhydrous CH₂Cl₂. The reaction was allowed to warm to room temperature overnight and diluted to 200 ml with CH₂Cl₂. The solution was washed with NaHSO₄, water, saturated NaHCO₃, brine and dried over MgSO₄. Concentration of the extract afforded crude tosylate 31 as a viscous oil, which was used in the next step without additional purification.

Sodium azide (2.3 g, 35.4 mmol) was added to a solution of crude tosylate 31 (˜20.8 mmol) in 100 ml of anhydrous DMF. The reaction was stirred at 50° C. for 5 h and then concentrated. The residual material was partitioned between water (100 ml) and ethyl acetate (200 ml). The organic phase was washed with saturated NaCl, dried over Na₂SO₄ and concentrated. The crude product was chromatographed on silica eluting with hexane/ethyl acetate. The product containing fractions were concentrated and dried under vacuum to afford 4.7 g (85%) of desired azide 32 as a colorless, viscous oil. ¹H NMR (DMSO-d6) δ 7.54 (ddd, J₁=7.5 Hz, J₂=1.2 Hz, J₃=1.2 Hz, 1H), 7.5-7.4 (m, 2H), 7.24 (ddd, J₁=8.4 Hz, J₂=2.7 Hz, J₃=1 Hz, 1H), 4.16 (m, 2H), 3.84 (s, 3H), 3.80 (m, 2H), 3.68 (t, J=5 Hz, 2H), 3.41 (t, J=5 Hz, 2H).

### N-[2-(2-[3-(Hydroxymethyl)phenoxy]ethoxy)ethyl](fluoren-9-ylmethoxy)carboxamide (34)

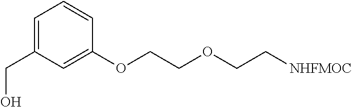

Lithium aluminum hydride (4.08 g, 107.5 mmol) was added to 100 ml of anhydrous THF under argon in three portions. The suspension was cooled to 0° C. (ice/water bath) and a solution of azide 32 (4.5 g, 17.0 mmol) in 40 ml of dry THF was added slowly (˜5 min) with stirring. The reaction was allowed to warm to room temperature and stirring was continued for 2 h. Excess LiAlH₄ was quenched by dropwise addition of water (20 ml) and the reaction mixture was concentrated to a semi-solid material. The solids were washed with 2-propanol until no product was detected in the washings (4×200 ml). Concentration of the extract afforded crude aminoalcohol 33 (3.6 g), which was utilized in the next step without additional purification.

To a stirred, cold (ice/water bath) solution of 33 (3.5 g, 16.5 mmol) in a mixture of THF (30 ml) and DMF (20 ml) was added N,N-diisopropylethylamine (2.76 ml, 15.8 mmol) followed by 9-fluorenylmethyl chloroformate (4.02 g, 15.54 mmol). The reaction was stirred at 0° C. for 30 min and concentrated. The obtained oil was partitioned between ethyl acetate and water. The organic phase was washed with brine, dried over Na₂SO₄ and concentrated. The resulting material was chromatographed on silica eluting with a gradient of ethyl acetate in hexane. Concentration of the pure product fractions afforded 6.5 g (88%) of 34 as a white solid. ¹H NMR (DMSO-d6) δ 7.89 (dd, J₁=7.2 Hz, J₂=1 Hz, 2H), 7.83 (dd, J₁=7.2 Hz, J₂=1 Hz, 2H), 7.42 (dt, J₁=7.4 Hz, J₂=1.2 Hz, 2H), 7.35 (dt, J₁=7.4 Hz, J₂=1.2 Hz, 2H), 7.21 (t, J=7.8 Hz, 1H), 6.87 (s+d, 2H), 6.79 (dd, J₁=8.7 Hz, J₂=1.8 Hz, 1H), 6.73 (t, J=5.4 Hz, 1H), 6.29 (s, 2H), 4.46 (s, 2H), 4.06 (m, 2H), 3.71 (t, J=4.8 Hz, 2H), 3.45 (m, 2H), 3.11 (m, 2H).

### N-[2-(2-[3-({[bis(methylethyl)amino](2-cyanoethoxy)phosphinooxy}methyl)phenoxy]ethoxy)ethyl](fluoren-9-ylmethoxy)carboxamide (35)

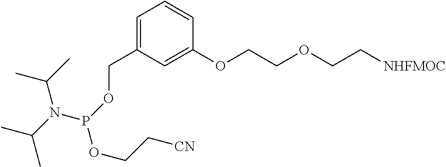

To a solution of 34 (1.0 g, 2.31 mmol) in 32 ml of anhydrous CH₂Cl₂ was added diisopropylammonium tetrazolide (0.55 g, 3.2 mmol) followed by 2-cyanoethyl N,N,N′N′-tetraisopropylphosphordiamidite (0.95 g, 3.15 mmol). The reaction was stirred overnight and diluted with 100 ml of ethyl acetate. The solution was washed with saturated NaHCO₃, NaCl and dried over Na₂SO₄. The extract was concentrated and the residue chromatographed on silica eluting with a gradient of ethyl acetate in hexane. Concentration of the pure product fractions and dried under vacuum afforded 1.1 g (75%) of 35 as a viscous oil. ¹H NMR (DMSO-d6) δ 7.89 (d, J=7.2 Hz, 2H), 7.69 (d, J=7.2 Hz, 2H), 7.40 (m, 2H), 7.32 (m, 2H), 7.26 (t, J=7.5 Hz, 1H), 6.92 (s+d, 2H), 6.84 (dd, J₁=7.5 Hz, J₂=2 Hz, 1H), 4.65 (m, 2H), 4.4-4.2 (m, 2H), 4.08 (m, 2H), 3.85-3.7 (m, 4H), 3.60 (m, 2H), 3.48 (t, J=6 Hz, 2H), 3.16 (m, 2H), 2.78 (t, J=6 Hz, 2H), 1.15 (m, 12H); ³¹P NMR δ 148.4 (s).

### Methyl 5-{2-[2-ethoxy]ethoxy}-3-(methoxycarbonyl)benzoate (36)

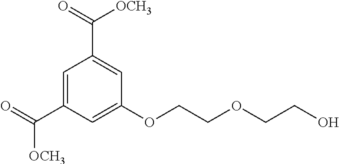

Diisopropylazodicarboxylate (2.6 g, 12.9 mmol) was added over 3 min to a stirred solution of dimethyl 5-hydroxyisophthalate (2.1 g, 10 mmol), diethylene glycol (1.2 g, 11.3 mmol) and triphenylphosphine (3.4 g, 13 mmol) in 50 ml of anhydrous THF. The reaction was stirred for 2 h and then concentrated. The resulting residue was suspended in 50 ml of ethyl ether and cooled to 0° C. Precipitated solids were removed by filtration and the filtrate was concentrated to a viscous liquid, which was then chromatographed on silica eluting with ethyl acetate. Concentration of the pure product fractions and drying under vacuum afforded 1.1 g (37%) of the title compound as a viscous oil. ¹H NMR (DMSO-d6) δ 8.07 (t, Hz, 1H), 7.69 (d, J=1.5 Hz, 2H), 4.63 (m, 1H), 4.23 (m, 2H), 3.89 (s, 6H), 3.78 (m, 2H), 3.51 (m, 2H), 3.50 (s, 2H).

### Methyl 5-{2-(2-azidoethoxy)ethoxy}-3-(methoxycarbonyl)benzoate (38)

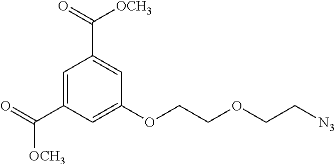

p-Toluene sulfonyl chloride (5.5 g, 28.8 mmol) was added in one portion to a stirred, cold (ice/water bath) solution of 36 (7.2 g, 24.1 mmol) in 75 ml of anhydrous pyridine. After being kept at 0° C. overnight the reaction was concentrated without using heating bath and the residue partitioned between ethyl acetate (200 ml) and 3N NaHSO₄ (200 ml). The aqueous phase was washed with extra amount of ethyl acetate and the combined organic washings were washed with saturated NaCl and dried over Na₂SO₄. Concentration of the extract afforded 9.5 g of crude tosylate 37 as a viscous oil, which was used in the next step without additional purification.

Sodium azide (4.0 g, 61.5 mmol) was added to a solution of crude tosylate 37 (9.5 g) in 220 ml of anhydrous DMF. The reaction was stirred at 50° C. for 5 h and then concentrated. The residual material was partitioned between water (100 ml) and ethyl acetate (200 ml). The organic phase was washed with saturated NaCl, dried over Na₂SO₄ and concentrated. The crude product was chromatographed on silica eluting with hexane/ethyl acetate. The product containing fractions were concentrated and dried under vacuum to afford 5.6 g (72%) of desired azide 38 as a colorless, viscous oil. ¹H NMR (DMSO-d6) δ 8.06 (s, 1H), 7.70 (s, 2H), 4.25 (t, J=4 Hz, 2H), 3.89 (s, 6H), 3.82 (t, J=4 Hz, 2H), 3.69 (t, J=5 Hz, 2H), 3.42 (t, J=5 Hz, 2H).

### N-[2-(2-[3,5-Bis(hydroxymethyl)phenoxy]ethoxy)ethyl](fluoren-9-ylmethoxy)carboxamide (40)

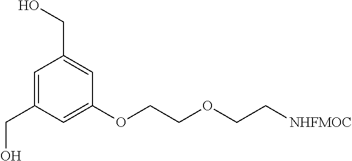

Lithium aluminum hydride (3.28 g, 86.4 mmol) was added to 100 ml of anhydrous THF under argon in three portions. The suspension was cooled to 0° C. (ice/water bath) and a solution of azide 38 (5.6 g, 17.3 mmol) in 40 ml of dry THF was added slowly (˜5 min) with stirring. The reaction was allowed to warm to room temperature and stirring was continued for another 2 h. Excess LiAlH₄ was quenched by dropwise (very slow at the beginning) addition of water (20 ml) and the reaction mixture was concentrated to a semi-solid material. Crude aminodiol was isolated from this material by extraction with 2-propanol and filtration. The solids were washed with additional 2-propanol until no product was detected in washings (4×200 ml). Concentration of the extract afforded crude aminodiol 39 (3.2 g), which was utilized in the next step without additional purification.

To a stirred, cold (ice/water bath) solution of crude amine 39 (3.2 g, 13.2 mmol) in 40 ml of DMF was added N,N-diisopropylethylamine (2.3 ml, 13.2 mmol) followed by 9-fluorenylmethyl chloroformate (3.4 g, 13.2 mmol). The reaction was stirred at 0° C. for 30 min. DMF was evaporated and the resultant oil was chromatographed on silica eluting with a gradient of MeOH 2.5-5% in CH₂Cl₂. Concentration of the product containing fractions afforded white solid which was then re-crystallized from CH₂Cl₂ to give 4.5 g (56%) of the title compound 40. ¹H NMR (DMSO-d6) δ 7.89 (d, J=7.4 Hz, 2H), 7.69 (d, J=7.2 Hz, 2H), 7.41 (t, J=7.4 Hz, 2H), 7.32 (t, J=7.2 Hz, 2H), 6.85 (s, 1H), 6.75 (s, 2H), 5.16 (t, J=5.8 Hz, 2H), 4.45 (d, J=5.8 Hz, 4H), 4.4-4.2 (m, 3H), 4.06 (t, J=4 Hz, 2H), 3.72 (t, J=4.2 Hz, 2H), 3.48 (t, J=6 Hz, 2H), 3.17 (m, 2H).

### N-(2-{2-(5-{[Bis(4-methoxyphenyl)phenylmethoxyl]methyl}-3-(hydroxymethyl)phenoxy) ethoxy}ethyl)(fluoren-9-ylmethoxy)carboxamide (41)

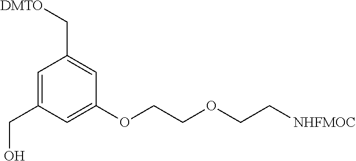

Dimethoxytrityl chloride (3.38 g, 10 mmol) was added in one portion to stirred, cold (ice/water bath) solution of diol 40 (4.4 g, 9.5 mmol) in 50 ml of anhydrous pyridine. The reaction was allowed to warm to room temperature. After being kept at room temperature for 5 h the reaction was concentrated and partitioned between ethyl acetate and cold 10% citric acid. The organic phase was washed with saturated NaCl and dried over Na₂SO₄. Desired mono-DMT substituted diol 41 was isolated from the mixture by silica gel column purification eluting with hexane/ethyl acetate. Concentration of the pure product fractions and drying under vacuum afforded 3.75 g (51%) of the title compound as an amorphous solid. ¹H NMR (DMSO-d6) δ 7.87 (d, J=7.3 Hz, 2H), 7.67 (d, J=7.2 Hz, 2H), 7.5-7.2 (m, 13H), 6.91 (d+s, 5H), 6.81 (s, 1H), 6.74 (s, 1H), 5.20 (t, J=6 Hz, 1H), 4.47 (d, J=6 Hz, 2H), 4.4-4.2 (m, 3H), 4.05 (m, 4H), 3.73 (s+m, 8H), 3.48 (t, J=6 Hz, 2H), 3.18 (m, 2H).

### 5-[N-(2-{2-(5-{[Bis(4-methoxyphenyl)phenylmethoxy]methyl}-3-(hydroxymethyl)phenoxy)ethoxy}ethyl)carbamoyl]-15-(2,2-dimethylpropanoyloxy)-1-oxospiro[3-hydroisobenzofuran-3,9′-xanthene]-12-yl 2,2-dimethylpropanoate (43)

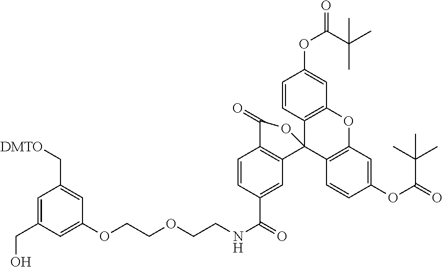

DBU (0.8 ml, 5.3 mmol) was added to a stirred solution of 41 (3.7 g, 4.83 mmol) in 50 ml of anhydrous CH₂Cl₂. After being stirred for 30 min the reaction mixture was concentrated and chromatographed on silica eluting with, first, CH₂Cl₂ to separate the protective group and, second, with 10:5:85 (MeOH:Et₃N:CH₂Cl₂) to elute the desired product. Solvent was evaporated and the residue dried under vacuum to afford 2.5 g (95%) of amine 42 as a semi-solid.

To a cold solution of the amine 42 (1.25 g, 2.3 mmol) and diisopropylethylamine (0.35 ml) in 30 ml of CH₂Cl₂ was added with stirring a cold (0° C.) solution of 1.42 g (2 mmol) of pentafluorophenyl dipivaloylfluorescein-6-carboxylate (29) (Nucleoside&Nucleotides (1997) 16(1&2), 107-114) in 10 ml of anhydrous THF. After being stirred at 0° C. for 1 h the reaction was warmed to room temperature and the solvent was evaporated. The resulting residue was chromatographed on silica eluting with 1:1 hexane/ethyl acetate followed with pure ethyl acetate. The pure product fractions were concentrated and dried to afford (2.2 g, 89% starting from 42) of 43 as a white, amorphous solid. ¹H NMR (DMSO-d6) δ 8.81 (t, J=5 Hz, 1H), 8.21 (d, J=8 Hz, 1H), 8.13 (d, J=8 Hz, 1H), 7.81 (s, 1H), 7.44 (s, 1H), 7.41 (s, 1H), 7.4-7.2 (m, 9H), 7.0-6.85 (m, 9H), 6.76 (s, 1H), 6.70 (s, 1H), 5.17 (t, J=6 Hz, 1H), 4.44 (d, J=6 Hz, 2H), 4.05 (m, 4H), 3.73 (s+m, 8H), 3.55 (t, J=5.4 Hz, 2H), 3.37 (m, 2H), 1.29 (s, 18H).

### 5-{N-[2-(2-[5-{[Bis(4-methoxyphenyl)phenylmethoxy]methyl}-3-({[bis(methylethyl)amino](2-cyanoethoxy)phosphinooxy}methyl)phenoxy]ethoxy)ethyl]carbamoyl}-15-(2,2-dimethylpropanoyloxy)-1-oxospiro[3-hydroisobenzofuran-3,9′-xanthene]-12-yl 2,2-dimethylpropanoate (44)

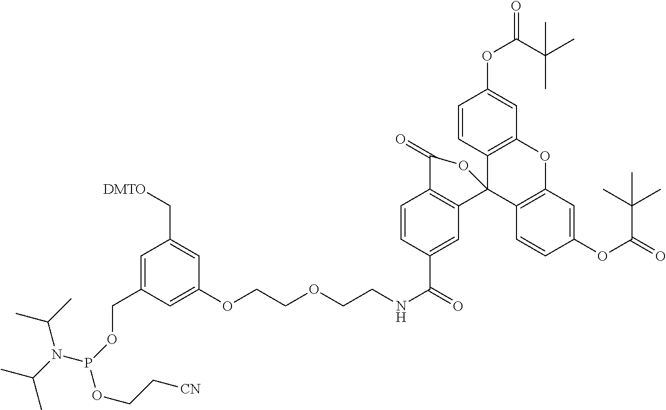

To a solution of 43 (2.1 g, 1.96 mmol) in 50 ml of anhydrous CH₂Cl₂ was added diisopropylammonium tetrazolide (0.43 g, 2.5 mmol) followed by 2-cyanoethyl N,N,N′N′-tetraisopropylphosphordiamidite (0.76 g, 2.5 mmol). The reaction was stirred overnight, concentrated and re-dissolved in ethyl acetate (100 ml). The solution was washed with saturated NaHCO₃, NaCl and dried over Na₂SO₄. The extract was concentrated to about 10 ml and slowly added into 300 ml of stirred pentane. The precipitated product was collected by filtration and dried under vacuum to afford 2.6 g (100%) of 44 as a white, amorphous solid. ¹H NMR (DMSO-d6) δ 8.79 (t, J=5 Hz, 1H), 8.21 (d, J=8 Hz, 1H), 8.13 (d, J=8 Hz, 1H), 7.80 (s, 1H), 7.44 (s, 1H), 7.41 (s, 1H), 7.4-7.2 (m, 9H), 7.0-6.8 (m, 9H), 6.78 (s, 1H), 6.71 (s, 1H), 4.65 (m, 2H), 4.02 (m, 4H), 3.74 (s+m, 12H), 3.56 (m, 2H), 3.38 (m, 2H), 2.75 (t, J=6 Hz, 2H), 1.29 (s, 18H), 1.14 (m, 12H); ³¹P NMR δ 148 (s).

### REFERENCES CITED

The following references, to the extent that they provide exemplary procedural or other details supplementary to those set forth herein, are specifically incorporated herein by reference.

### U.S. AND FOREIGN PATENT DOCUMENTS

U.S. Pat. No. 5,656,430;
U.S. Pat. No. 5,763,178;
U.S. Pat. No. 6,340,566;
U.S. Pat. No. 5,955,268;
U.S. Pat. No. 5,516,663;
U.S. Pat. No. 5,792,607;
U.S. Pat. No. 7,252,940;
U.S. Pat. No. 5,574,142;
U.S. Pat. No. 5,925,744;
U.S. Pat. No. 7,381,818;
U.S. Pat. No. 4,683,202;
U.S. Pat. No. 5,409,818;
U.S. Pat. No. 4,957,858;
U.S. Pat. No. 5,124,246;
U.S. Pat. No. 5,011,769;
U.S. Pat. No. 5,422,252;
European Patent Application No. 1136569; and
International Patent Publication WO 93/20191

### NONPATENT REFERENCES

Prokaryotic Base Excision Repair, Wilson 111, D. M., Engelward, B. P. and Samson, L. (1998) pp. 29-64; from DNA Damage and Repair, V.1: DNA Repair in Prokaryotes and Lower Eukaryotes, Edited by: J. A. Nickoloff and M. F. Hoekstra, Humana Press Inc., Totowa, N.J.
Regulation of Endonuclease IV as Part of an Oxidative Stress Response in Escherichia coli, Weiss B. (1998) pp. 85-96; from DNA Damage and Repair, V.1: DNA Repair in Prokaryotes and Lower Eukaryotes, Edited by: J. A. Nickoloff and M. F. Hoekstra, Humana Press Inc., Totowa, N.J.
Kaboev, O. K., Luchkina, L. A., and Kuziakina, T. I., J. Bacteriol. 164: 878-881, 1985.
Warner, H. R., J. Bacteria 154: 1451-1454, 1983
Brian J. Haas, B. J., J. Bacteriol. 181: 2834-2839, 1999
Bibor, V., and Verly, W. G., J. Biol. Chem. 253: 850-855, 1978
Barany, PCR Methods and Applications 1: 5-16, 1991
Vielle and Zeikus, Microbial. Molec. Biol. Reviews, 65: 1-43, 2001
Sartori and Jiricny, J. Biol. Chem., 278: 24563-24576, 2003
Back et al., Biochem. Biophys. Res. Comm., 346: 889-95, 2006
Vielle & Zeikus, Microbial. Mol, Biol. Rev., 65:1-43, 2001
Giver et al., PNAS 95, 12809-12813, 1998
Veile and Zeikus, Trends in Biotech., 17: 135-136, 1999
Van den Berg et al., PNAS 95: 2056-2060, 1998
Veile and Zeikus, Nat. Struct. Biol., 5:470-475, 1998
Kutyavin, I V., et al. Nucleic Acids Research 34:19, 2006
Nelson et al. Nucleosides & Nucleotides 16(10 & 11), 1997
Jadhav et al. Nucleoside & Nucleotides 16(1&2), 107-114, 1997

What is claimed:

1. A method of detecting a target nucleic acid in a sample, comprising:
a) contacting the sample with at least one endonuclease IV substrate probe such that the endonuclease IV substrate probe hybridizes to the target nucleic acid to form a reaction mixture, wherein the endonuclease IV substrate probe comprises an oligonucleotide sequence (NA) attached at a 3′ end via a phosphodiester bond of a phosphate group, to a functional tail (R), comprising a hyperthermostable endonuclease IV substrate;
b) contacting the reaction mixture with a hyperthermostable endonuclease IV;
c) incubating the reaction mixture under reaction conditions sufficient to allow the hyperthermostable endonuclease VI to cleave the phosphodiester bond; and
d) detecting the reporter group on the cleaved functional tail (R), whereby the target nucleic acid is detected,
wherein the hyperthermostable endonuclease VI preferentially cleaves the phosphodiester bond attaching the functional tail (R) to the oligonucleotide sequence (NA) when the oligonucleotide sequence (NA) is hybridized with a complementary target nucleic acid sequence in comparison to when the oligonucleotide sequence (NA) is unhybridized or hybridized to a non-complementary target nucleic acid.

2. The method of claim 1, wherein the hyperthermostable endonuclease IV has been
isolated from: Aquifex pyrophilus, Thermocrinus rubber, Thermotoga maritime, Thermotago strain FjSS3-B1, Sulfolobus shibatae, S. solfataricu, Slygiolabus azoricus, Acidianus infernus, A. ambivalens, Thermoproteus tenax, T. neurtophilus, T. uzoniensis, Pyrobaculum islandicum, P. organotrophum, P. aerophilum, Thermojilum pendens, Desulfurococcus mobilis, D. amylolyticus, Staphylothermus marinus, Thermosphaera aggregans, Pyrodictium occultum, P. abyssi, P. prockii, Hyperthermus butylicus, Thermodiscus maritimus, Pyrolobus fumarii, Aeropyrum pernix, Caldococcus litoralis, Palaeococcus ferrophilus, Thermococcus aggregans, T barophilus, T. guaymasensis, T. celler, T. acidaminovorans, T. chitonophagus, T. barossii, T. litoralis, T. profundus, T. hydrothermalis, Pyrococcus furiosus, P. woesei, P. abyssi, P. horikoshii, Archaeoglobus fulgidus, A. profundus, Methanococcus jannaschii, M. valcanius, M. vervens, M. igneus, M. infernus, Methanothermus fervidus, M. sociabilis, or Methanopyrus kandleri.

3. The method of claim 1, wherein the hyperthermostable endonuclease IV has been obtained using protein engineering.

4. The method of claim 1, wherein the endonuclease IV substrate probe further comprises a quencher.

5. A hyperthermostable endonuclease IV substrate probe for detection of nucleic acid amplification, comprising:
an oligonucleotide sequence (NA),
wherein the oligonucleotide sequence (NA) is attached at a 3′ end via a phosphodiester bond of a phosphate group to a functional tail (R), comprising a hyperthermostable endonuclease IV substrate, and
wherein a hyperthermostable endonuclease VI preferentially cleaves the phosphodiester bond attaching the functional tail (R) to the oligonucleotide sequence (NA) when the oligonucleotide sequence (NA) is hybridized with a complementary target nucleic acid sequence in comparison to when the oligonucleotide sequence (NA) is unhybridized or hybridized to a non-complementary target nucleic acid.

6. The hyperthermostable endonuclease IV substrate probe of claim 5, wherein the probe has a structure of: 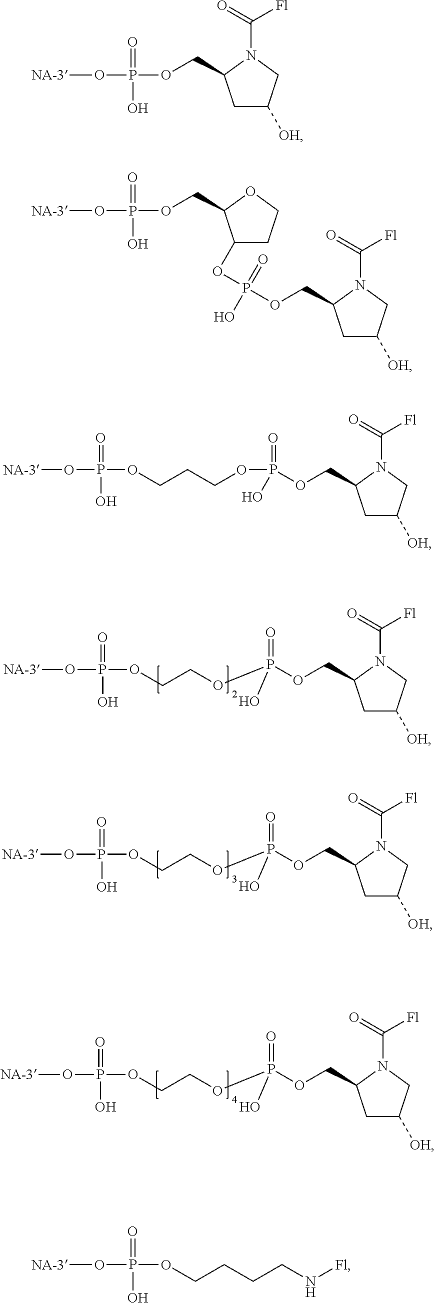 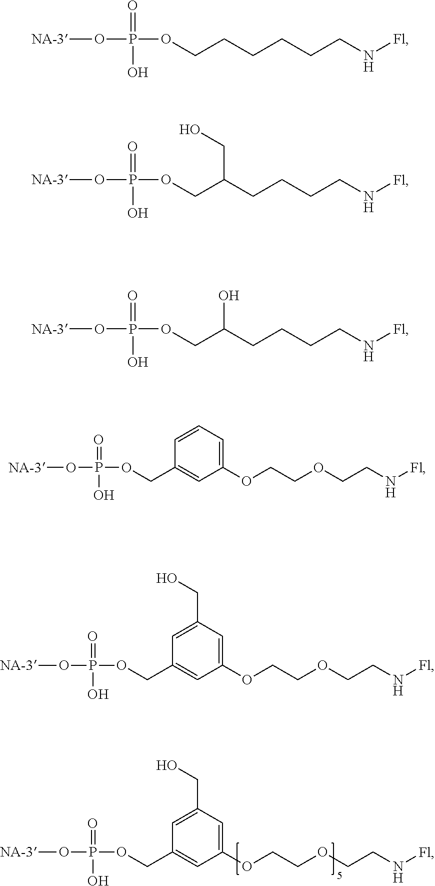
wherein F1 is a detectable label.

7. The hyperthermostable endonuclease IV substrate probe of claim 5, wherein the probe has a structure of: 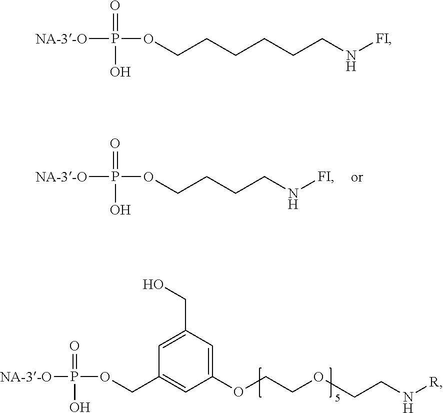
wherein F1 is a detectable reporter group.

8. The hyperthermostable endonuclease IV substrate probe of claim 5, wherein the probe further comprises a quencher.

9. The hyperthermostable endonuclease IV substrate probe of claim 5, wherein the functional tail further comprises a linker (L).

10. The hyperthermostable endonuclease IV substrate probe of claim 9, wherein the linker (L) contains between 1 and 40 main chain atoms, and where in the main chain atoms are selected from the group consisting of: C, O, N, S, and P.

11. The hyperthermostable endonuclease IV substrate probe of claim 9, wherein the linker (L) includes saturated or unsaturated ring structures.

12. The hyperthermostable endonuclease IV substrate probe of claim 5, wherein the functional tail further comprises a detectable reporter group.

13. The hyperthermostable endonuclease IV substrate probe of claim 12, wherein the detectable reporter group is selected from the group consisting of: mass tags, fluorescent dyes, non-fluorescent dyes, radioisotopes, functional ligands like biotin, oligopeptides, and carbohydrates.
